(19) United States
(12) Patent Application Publication
(10) Pub. No.: US 2012/0121509 A1
(43) Pub. Date: May 17, 2012

(54) VITAL FLUOROCHROME CONJUGATES AND METHODS OF USE

(75) Inventors: Lee Josephson, Reading, MA (US); Elisabeth Garanger, Bordeaux (FR); Scott Hilderbrand, Swampscott, MA (US); David Sosnovik, Somerville, MA (US); Hushan Yuan, West Roxbury, MA (US)

(73) Assignee: THE GENERAL HOSPITAL CORPORATION, Boston, MA (US)

(21) Appl. No.: 13/376,043

(22) PCT Filed: Jun. 4, 2010
(86) PCT No.: PCT/US2010/037425

§ 371 Date: Jan. 20, 2012

Related U.S. Application Data
(60) Provisional application No. 61/184,523, filed on Jun. 5, 2009.

(51) Int. Cl. A61K 51/04; A61K 49/10; C07F 5/00; C07D 417/06; C07F 1/08; C07D 417/14

(52) U.S. Cl. 424/1.89; 546/10; 546/175; 534/16; 546/5; 424/9.361; 424/9.3; 424/1.65

(57) ABSTRACT

The present invention provides compositions and methods based on vital fluorochrome conjugates that are useful for imaging dying and dead cells.

### CROSS REFERENCE TO RELATED APPLICATION

This application claims priority to U.S. Provisional Patent Application No. 61/184,523, filed on Jun. 5, 2009, the disclosure of which is incorporated herein by reference in its entirety.

### STATEMENT REGARDING FEDERALLY SPONSORED RESEARCH OR DEVELOPMENT

The present invention was made with government support Under Grant Nos. R01-EB004472 and R01-EB00066 awarded by the National Institutes of Health. The United States Government has certain rights in the invention.

### FIELD OF THE INVENTION

The present disclosure describes the design and synthesis of vital fluorochrome conjugates for the imaging of dead or dying cells.

### BACKGROUND OF THE INVENTION

Cells often die in well-defined stages, transitioning from healthy (vital) cells to apoptotic cells and then to necrotic cells. An important class of compounds for imaging necrosis (cell death) is vital fluorochromes (VF's). VF's are widely used to visualize necrosis (or cell viability by their exclusion from viable cells) by fluorescence microscopy or fluorescence activated cell sorting (FACS). Vital fluorochromes are often used in conjunction with fluorescent annexin V's, which bind to both apoptotic and necrotic cells. Thus by using VF's and annexin V (and two spectrally distinct fluorochromes), three states of cells can be defined (vital, apoptotic, and necrotic). VF's can be used to visualize cell death in biological research, but cannot be used to visualize death/necrosis by fluorescence imaging in humans, because the distances light must traverse through tissues in these methods lead to massive attenuation for organisms of this size.

There are diverse needs for imaging apoptosis/necrosis in medicine. These include (i) determining functional cell mass (and organ health) in diabetes (ii) predicting organ rejection, (iii) distinguishing apoptosis from necrosis in ischemia/reperfusion injury, (iv) imaging post chemotherapy or post radiation therapy levels of tumor apoptosis/necrosis as an indication of a therapeutic response, and (v) imaging vulnerable plaque in blood vessels.

A wide variety of agents have been employed to image apoptotic or necrotic cells. However, some agents typically used to image apoptosis or necrosis suffer from a lack of specificity for imaging cell death, e.g., because they are retained by healthy cells of the liver, spleen, or other tissues. Some agents lack a molecular target or clear mechanism for retention by apoptotic and/or necrotic cells, but not by vital, healthy cells. In some cases, the agents are not fluorescent, so their uptake in individual cells, which maybe vital or apoptotic or necrotic, cannot be compared with reference fluorescent probes to determine cell health. As a result of these challenges there is no currently approved imaging agent for imaging either apoptosis or necrosis. Thus, there exists a need for a fluorescent agent that can image cell death in specific cells in a research or commercial setting, with a well-defined mechanism of action/molecular target, and that can be used with established clinical imaging modalities.

### SUMMARY OF THE INVENTION

The present invention provides, inter alia, compositions and methods of making and using vital fluorochrome conjugates that can be used to image dead or dying cells resulting from diseases such as diabetes, organ transplant rejection, myocardial infarction, responses to treatments such as chemotherapy or radiation therapy, and plaques in blood vessels. The vital fluorochrome conjugates include a vital fluorochrome, a reporter group, e.g., one that is capable of attaching to a detectable metal, halide or other atom, and a linker connecting the vital fluorochrome to the reporter group. The vital fluorochrome binds to nucleic acids in dead or dying cells and provides the specificity for these cells rather than healthy cells. The reporter groups can be detected by various imaging modalities including, e.g., single photon emission computed tomography (SPECT) scan, positron emission tomography (PET) scan, or magnetic resonance imaging (MRI) scan or other imaging modality.

Accordingly, in a first aspect the present invention provides compositions for imaging dead or dying cells. The compositions include a vital fluorochrome conjugate including a vital fluorochrome, a reporter group, and a linker that connects the fluorochrome to the reporter group.

In some embodiments, the vital fluorochrome includes an aliphatic arm attached to an unsaturated ring system at a first end of the aliphatic arm, and a quaternary, positively charged nitrogen attached to the aliphatic arm at a second end.

In some embodiments, the vital fluorochrome conjugate comprises a compound of Formula I:

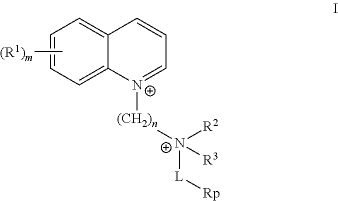

wherein:

L is a linker and comprises a C₂₋₂₀ alkyl chain, wherein any of the carbons in the C₂₋₂₀ alkyl chain can be replaced with —C(O)—, —C(O)₂, O, S, S(O), S(O)₂, —NR^{a}—, —NR^{a}C(O)—, or a triazole;

Rp is a reporter group;

R¹ is aryl or heteroaryl optionally substituted by aryl or heteroaryl; or 1 or 2 R¹ adjacent to each other and together with the carbon atoms to which they are attached form 1 or 2 aryl or heteroaryl rings, optionally substituted by 1, 2, 3, or 4 substituents independently selected from OR^{a} and —NR^{a}₂;

R² and R³ are independently selected from H and C₁₋₆ alkyl; or

R² and R³ are each independently L-Rp;

R^{a} is selected from H and C₁₋₆ alkyl; and

m and n are each independently 0, 1, 2, 3, 4, 5, 6, 7, 8, 9, or 10.

In certain embodiments, the reporter group is a metal chelator that chelates a detectable metal atom. In some embodiments, the metal is paramagnetic or radioactive. In some embodiments, the metal is selected from gadolinium, dysprosium, ¹¹¹indium, ⁹⁹mTc, ⁶⁴copper, and ⁶⁸gallium.

In some embodiments, the reporter group is

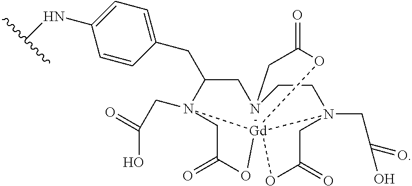

In some embodiments, the reporter group comprises a radioactive halide, such as, for example, ¹⁸Fluorine.

In a further embodiment, the reporter group is

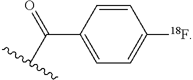

In a further embodiment, the vital fluorochrome conjugate is selected from:

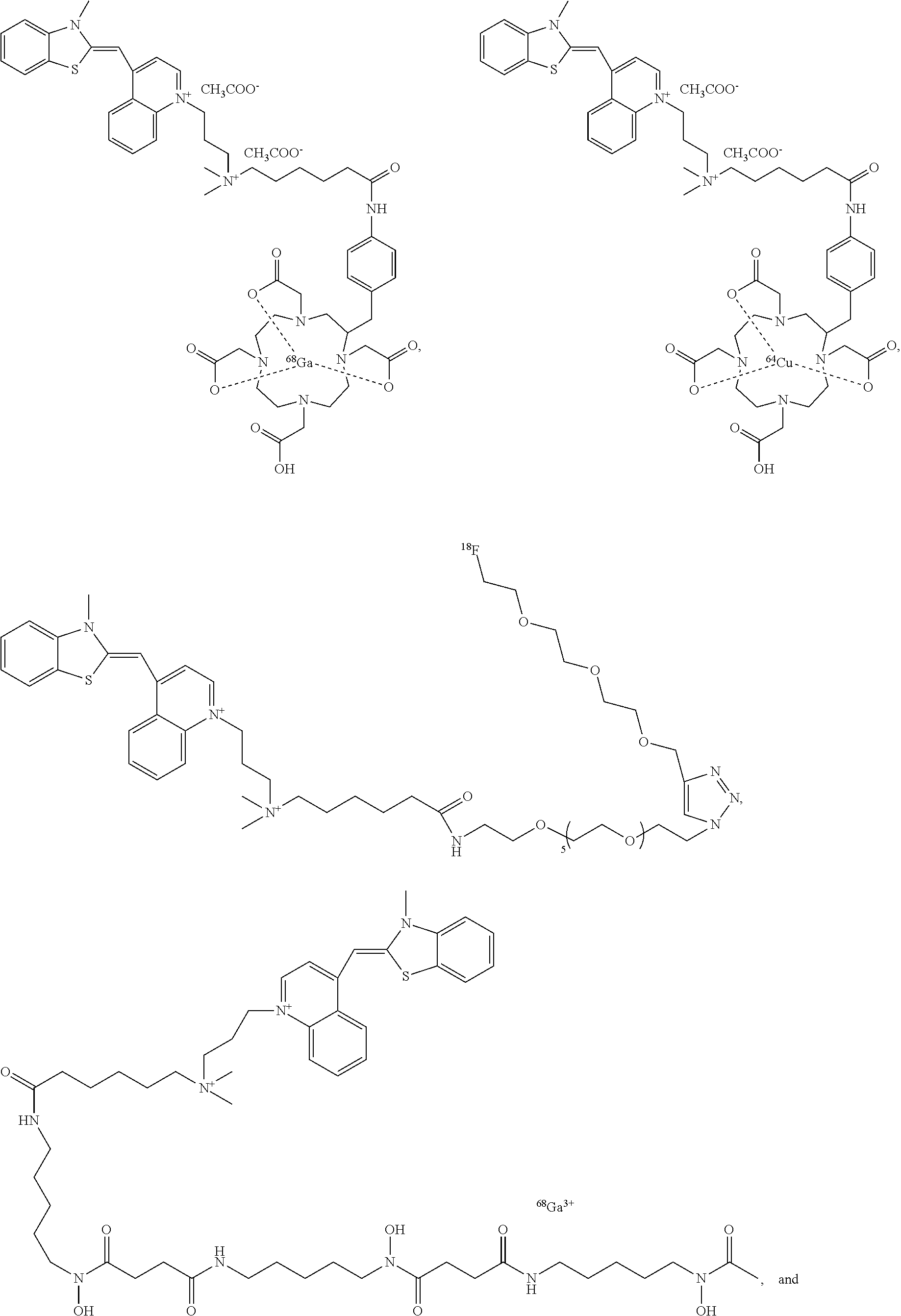

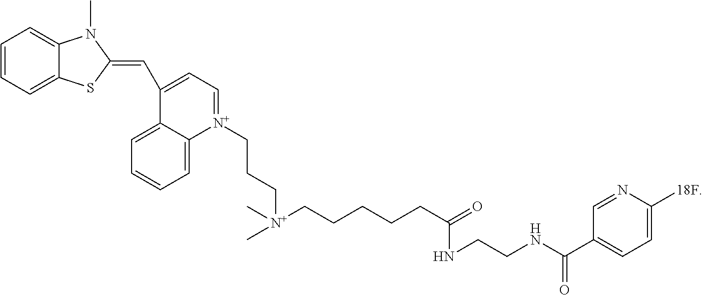

In another aspect, the invention provides methods of imaging dead or dying cells in a subject. These methods include administering to the subject a vital fluorochrome conjugate that includes a vital fluorochrome, a reporter group, and a linker that connects the vital fluorochrome to the reporter group, for a time sufficient for the vital fluorochrome conjugate to enter dead or dying cells and bind to nucleic acids; and obtaining an image of the vital fluorochrome conjugate in the subject.

In one embodiment, the image is obtained by a single photon emission computed tomography (SPECT) scan, a positron emission tomography (PET) scan, or a magnetic resonance imaging (MRI) scan.

In some embodiments, the subject is a mammal. In certain embodiments, the dead or dying cells are cancer cells treated by chemotherapy or cells from an organ transplanted into the subject.

In another aspect, the invention provides methods of detecting dead or dying cells in myocardium tissue in a subject. These methods include administering to the subject a vital fluorochrome conjugate that includes a vital fluorochrome, a reporter group, and a linker that connects the vital fluorochrome to the reporter group, for a time sufficient for the vital fluorochrome conjugate to enter the dead or dying myocardial cells and bind to nucleic acids; and obtaining an image of the vital fluorochrome conjugate in the subject.

In certain embodiments, the tissue is ischemic due to a myocardial infarction. In other embodiments, the dead or dying cells are imaged between 1 day and 10 days after a myocardial infarction occurs, e.g., within 24 hours, within 24 to 72 hours, or over 72 hours. In other embodiments, the methods are used to determine when the myocardial infarct occurred, and, optionally, determining or selecting a specific therapy based on this information.

In yet another aspect, the invention provides methods of preparing compounds of Formula I:

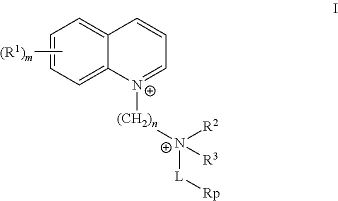

wherein L, Rp, R¹, R², R³, R^{a}, m, and n are each as defined herein.

The methods include (a) reacting a compound of Formula I-(1):

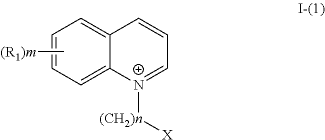

wherein R¹, m, and n are each as defined herein; and X is a halide;

with an amine of Formula I-(2):

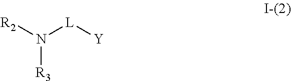

wherein R² and R³ are each as defined herein; and

Y is selected from

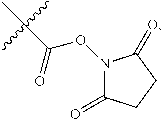

### —NH₂, N₃, and

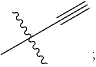

to provide a compound of Formula I-(3):

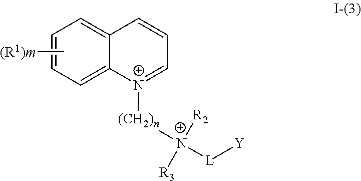

wherein L, Y, R¹, R², R³, m, and n are each as defined herein; and

(b) reacting the compound of Formula I-(3) with a compound of Formula I-(4):

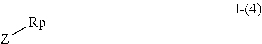

wherein Rp and Y are as defined herein; and

Z is selected from

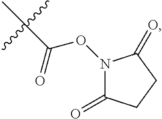

### —NH₂, N₃, and

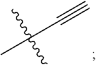

to produce the compound of Formula I.

In some embodiments, Y is N₃ and Z is

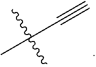

In other embodiments, Y is

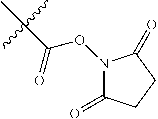

### and Z is NH₂.

### DEFINITIONS

As used herein, “alkyl” refers to a hydrocarbon chain that may be a straight chain or branched chain, containing the indicated number of carbon atoms. For example, C1-C12 alkyl indicates that the group may have from 1 to 12 (inclusive) carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, “heteroaryl” groups refer to aromatic heterocycles having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, and indolinyl. In some embodiments, the heteroaryl group has from 1 to about 20 carbon atoms. In some embodiments, the heteroaryl group contains 3 to about 14 ring-forming atoms. In other embodiments, the heteroaryl group has 1 to about 4 heteroatoms.

As used herein, the term “aliphatic arm” refers to a hydrocarbon chain that is a straight chain or branched chain, e.g., containing from 2 to 20 carbon atoms. In some embodiments, the aliphatic arm contains 2 to 10 carbon atoms.

By virtue of their design, the vital fluorochrome conjugates described herein possess certain advantages and benefits. First, the vital fluorochrome conjugates can be used to distinguish healthy cells from those that are dead or dying. Second, the vital fluorochrome conjugates enable the imaging of dead or dying cells through a variety of widely used imaging modalities (PET, SPECT, and MRI), while at the same time being fluorescent. In addition, vital fluorochrome conjugates can be used to assess the health of ischemia damaged myocardium, which can indicate the extent of damage to the myocardium or the potential for salvaging the damaged myocardium.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A and 1B are images of vital fluorochromes.

FIG. 2A is a bar graph depicting binding of gadolinium thiazole orange (GadoTO), which is an MR contrast agent for the detection of cell death that includes a nucleic acid-binding fluorophore attached to a gadolinium chelate, to DNA as monitored by relaxometry.

FIG. 2B is a graph depicting binding of GadoTO to DNA as monitored by fluorescence.

FIG. 3 is a graph depicting a dual wavelength fluorescence activated cell sorter showing the failure of GadoTo to bind to healthy Jurkat cells, but the ability to bind to necrotic cells.

FIG. 4 is a pair of related graphs depicting parallel binding of GadoTO and Propidium Iodide (PI) to Jurkat T cells as imaged by single channel FACS.

FIG. 5A is a diagram showing the interaction of GadoTO with cells induced to become necrotic by a prolonged ischemia or prolonged camptothecin (CPT) treatment.

FIG. 5B is a magnetic resonance (MR) image of packed cell phantoms of camptothecin treated Jurkat cells.

FIGS. 5C and 5D are MR images depicting ischemia induced necrosis in a gated image of a beating mouse heart in a gradient echo and an inversion recovery, respectively.

FIGS. 6A and 6B are MR images depicting a beating mouse heart imaged during phase 2 and phase 4 of the post myocardial infarction reaction phases, respectively.

### DETAILED DESCRIPTION

The present invention provides compositions and methods for imaging dead or dying cells in vivo and in vitro. For example, the cells may be dying as a result of diabetes, organ transplant rejection, myocardial infarction, chemotherapy or radiation therapy, or a plaque in a blood vessel. These methods include the use of a vital fluorochrome conjugate that includes a vital fluorochrome, a reporter group, and a linker connecting the vital fluorochrome to the reporter group. The vital fluorochrome conjugate can pass through the permeable membrane of dead or dying cells, but not the intact membrane of healthy cells, and can be detected by SPECT, PET or MRI, while at the same time being fluorescent.

### Structure of the Vital Fluorochrome Conjugates

The vital fluorochrome conjugates include a vital fluorochrome, a reporter group, e.g., a group capable of attaching to a detectable group such as a metal, halide, or other atom or atoms, and a linker connecting the fluorochrome to the reporter group.

### Vital Fluorochromes

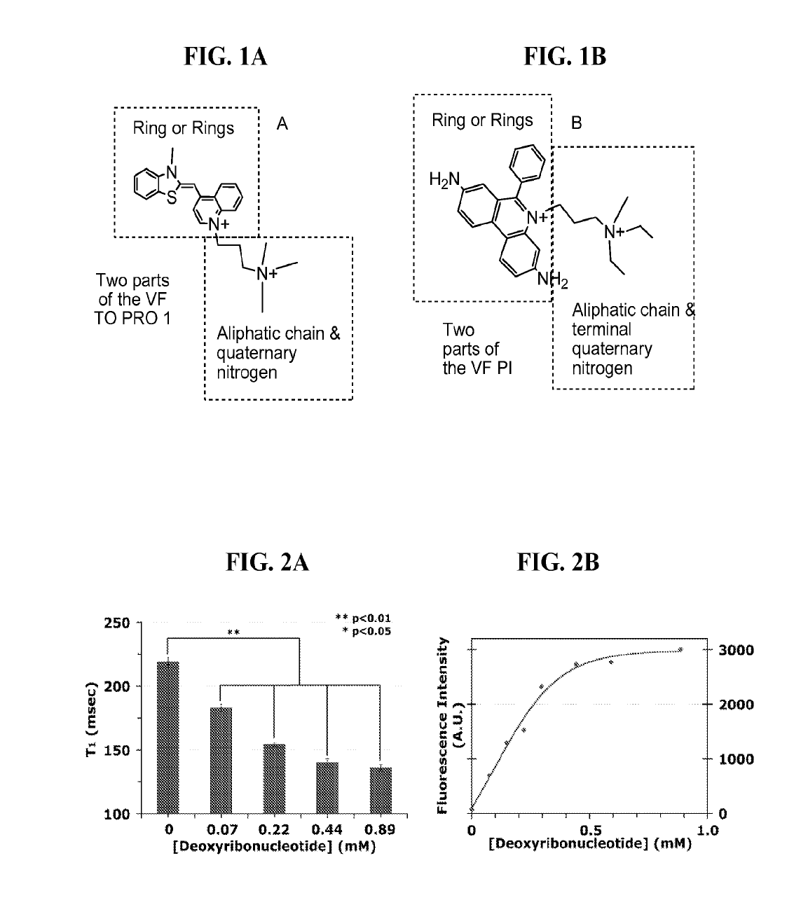

The vital fluorochromes (VF) are positively charged, low molecular weight organic compounds, e.g., less than about 800 Daltons, and contain two parts: 1) an unsaturated ring or rings and, 2) an aliphatic arm. As shown in FIGS. 1A and 1B, the aliphatic arm has two ends, and its first end is attached to the unsaturated ring or rings and its second end is attached to a terminal quaternary nitrogen that is positively charged. The positive charge assists in preventing the VFs from passing through intact membranes in healthy cells and in their binding to nucleic acids. The unsaturated ring or rings of the vital fluorochromes give rise to absorption and emission (Abs/em) of light (fluorescence) and are shown in Table 1 below. The rings intercalate between the bases of double-stranded DNA. VFs can also show weaker binding to single-stranded nucleic acids. In addition to playing a role in making the VF membrane impermeable, positive charges also assist in tight DNA binding, by interacting with negative phosphate groups on DNA.

TABLE 1
VFs and their Optical Properties

| VF | VF Structure | Abs/em |
|---|---|---|
| TO-PRO 1 | 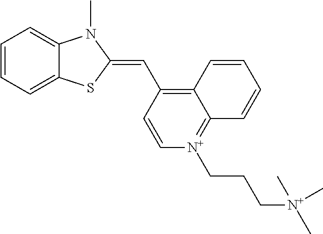 | 515/531 (nm) |
| TO-PRO 3 | 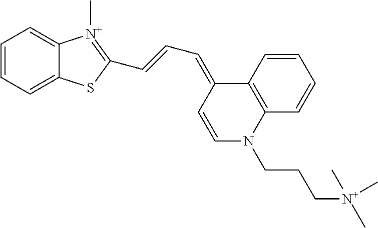 | 612/631 |
| TO-PRO 5 | 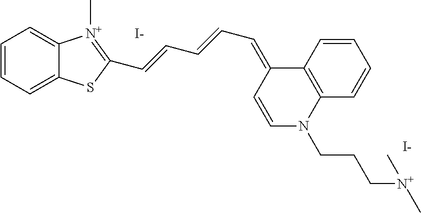 | 745/770 |
| YO-PRO | 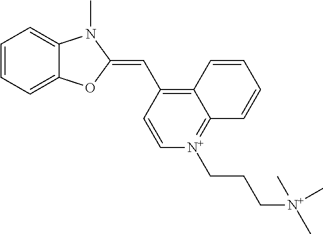 | 491/509 |
| YO-PRO 3 | 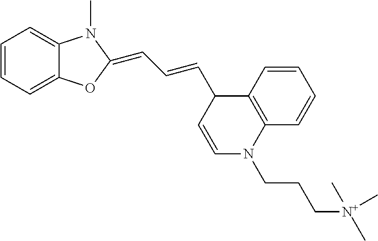 | 612/631 |
| Propidium Iodide (PI) | 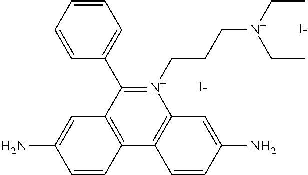 | 530/625 |
| Sybr Green | 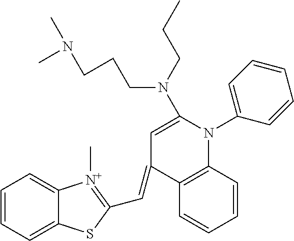 | 490/520 |

In addition, vital fluorochromes must 1) selectively bind to dead or dying cells, 2) permit the attachment of a reporter group, e.g., a chelate or other chemical group that can covalently bond to a detectable atom, and 3) permit the attachment of a chelate or other chemical group without changing the selective permeability for dead or dying cells or their ability to bind to nucleic acids. In addition, to attach a reporter group to a VF, to obtain a vital fluorochrome conjugate, critical features of the VF must be maintained: 1) the ring structure must not be modified and 2) the amount, distribution, and type of positive charge should be maintained.

Many fluorochromes do not possess the properties of vital fluorochromes (e.g., due to impermeability to the membranes of healthy cells, retention by binding nucleic acids). Some fluorochromes are chemically reactive and covalently modify amino acids in vivo or in vitro. These include fluorochromes featuring isothiocyanates (e.g., fluoroscein), the NHS esters (rhodamine, fluorescein, Cy5.5. Cy3.5 and halide leaving groups (e.g. NBD-Cl, Dansyl chloride). Other fluorochromes have negatively rather than positively charged groups (Cy5.5, Cy3.5). Some fluorochromes, such as 4′,6-diamidino-2-phenylindole (DAPI) and bisbenzimide (Hoescht 33342), bind to DNA, but penetrate the membranes of intact, healthy cells as well, and thus lack the requisite selectivity.

### Linkers

The term “linker” as used herein refers to a group of atoms, e.g. 5-100 atoms, and may be comprised of the atoms or groups, carbon, amino, alkylamino, oxygen, sulfur, sulfoxide, sulfonyl, amide, and carbonyl. The linker is connected at a first end to the vital fluorochrome through the quaternary nitrogen on the aliphatic arm. Thus, in order for the linker to be installed onto the VF and yield a quaternary nitrogen, the linker must have an amino group on the first end (see intermediate 2 in Scheme 1) which can displace the halide intermediate (see intermediate 1 in Scheme 1) and produce the quaternary nitrogen. In addition, the linker is connected at a second end to the reporter group through a covalent bond. Examples of covalent bonds include, but are not limited to an amide bond or a triazole ring. Alternatively, a first linker can be extended with a second linker, to produce a longer and more complex linker, prior to attachment of the reporter group. Examples of reporter groups, linkers, and vital fluorochrome are shown in Table 2 below.

TABLE 2
VF-Linker-Reporters of Vital Fluorochromes Conjugates
Summary of examples and additional possibilities

| | Vital | |
|---|---|---|
| | Fluoro- | |
| | chrome, VF | Linker |
| Ex 1 | 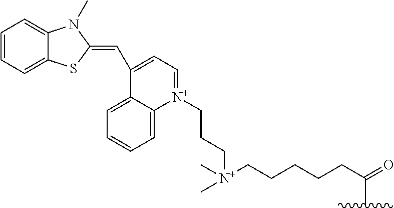 | 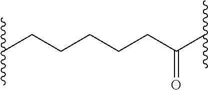 |
| Ex 2 | 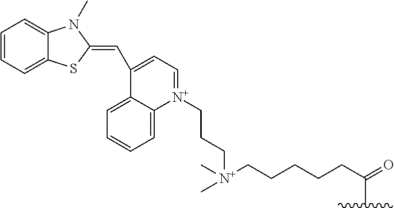 | 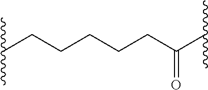 |
| Ex 3 | 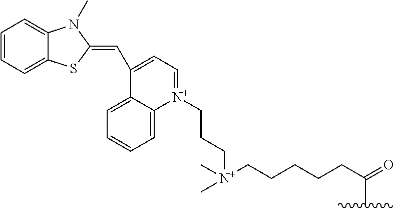 | 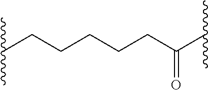 |
| Ex 4 | 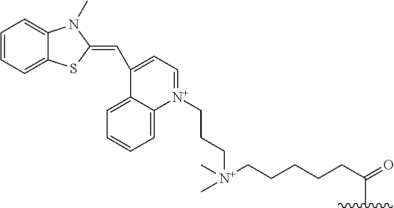 | 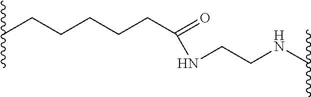 |
| Ex 5 | 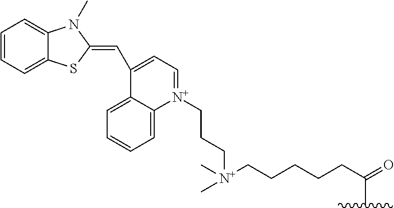 | 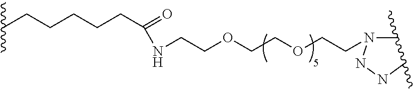 |
| Ex 6 | 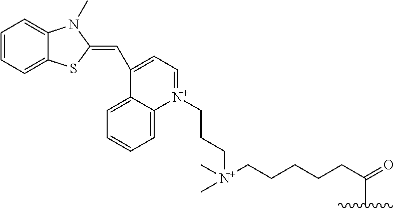 | 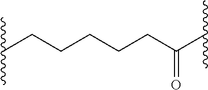 |
| Ex 7 | 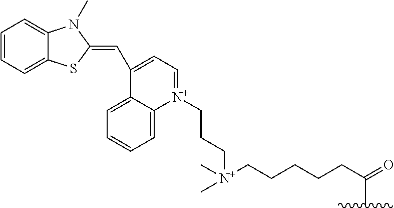 | 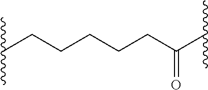 |
| Ex 8 | 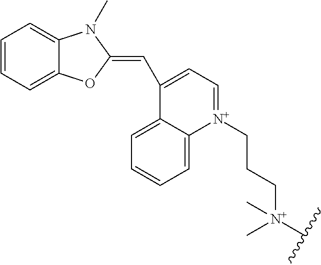 | 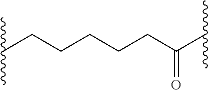 |
| Ex 9 | 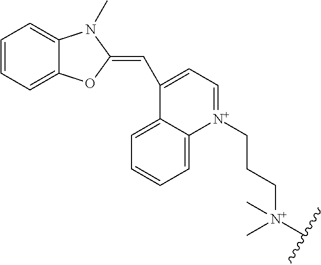 | 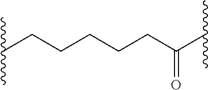 |
| Ex 10 | 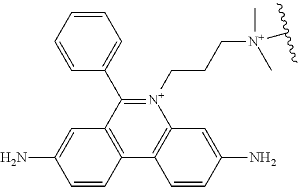 | 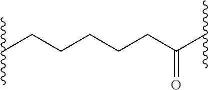 |
| Additonal | | |
| Reporters | | |
| Additional | | |
| Reporters | | |

| | Reporter Group |
|---|---|
| Ex 1 | 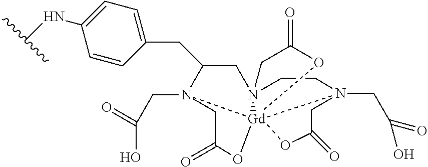 |
| Ex 2 | 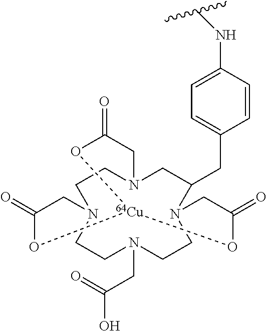 |
| Ex 3 | 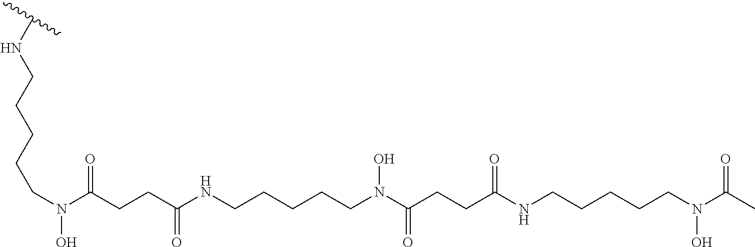 |
| Ex 4 | 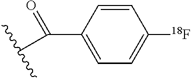 |
| Ex 5 | 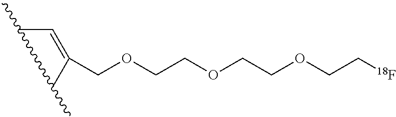 |
| Ex 6 | 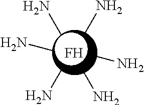 |
| Ex 7 | 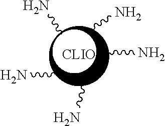 |
| Ex 8 | 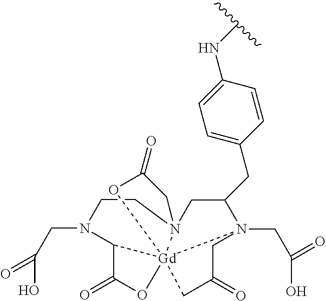 |
| Ex 9 | 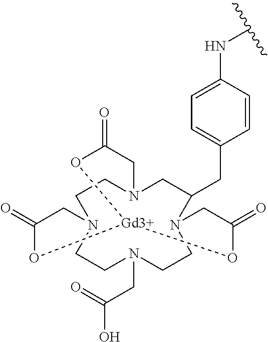 |
| Ex 10 | 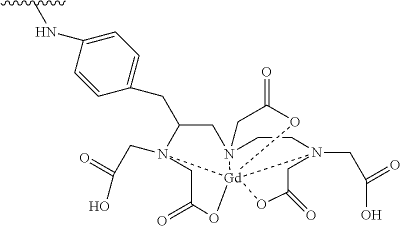 |
| Additional | pNH2-CHX-A-DTPA |
| Reporters | pNH2BnPCTA |
| | pNH2Bn-oxo-DO3A |
| | (from Macrocyclics) |
| Additonal Reporters | 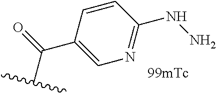 |

### Reporter Groups

Reporter groups consist of structures which are detectable, e.g. a group capable of attaching to a detectable atom such as a paramagnetic or radioactive metal, or a radioactive halide, or other detectable atom. The reporter groups must have a reactive group that can form a covalent bond with the linker. The paramagnetic or radioactive metal can be attached to the reporter group structure by chelating to the reporter group. Exemplary paramagnetic metals include Dy and Gd and radioactive metals include, but are not limited to ¹¹¹In, ⁹⁹ mTc, ⁶⁴Cu, and ⁶⁸Ga. Non metals like radioactive halides or ¹¹C can also be used. Table 3 below demonstrates various metals and non metals that can be used as part of a reporter group, their notable properties, and their method of detection. Examples of radiolabeled atoms include, but are not limited to ¹⁸F, ¹⁴C, and ³H. Superparamagnetic cross-linked iron-oxide (CLIO) particles activated by reaction with ammonia to create an amine handle on the CLIO particle can be used to attach the linker carrying a carboxylic acid or ester reactive group.

TABLE 3
Reporter Atoms that can be used with reporter groups

| | Imaging |
|---|---|
| Reporter Atom | Modality |
| Dy | MRI |
| Gd | MRI |
| Iron (superparamagnetic | MRI |
| iron oxide) | |
| ¹¹¹In | SPECT |
| ⁶⁴Cu | PET |
| ⁶⁸Ga | PET |
| ¹⁸F | PET |
| ¹²³I | SPECT |
| ¹²⁴I | PET |
| ⁸⁹Zr | PET |
| ⁸⁶Yt | PET |
| ¹¹C | PET |

### Synthesis of the Vital Fluorochrome Conjugates

A series of vital fluorochrome conjugates are prepared by the methods outlined below in Scheme 1. Unsaturated ring intermediate 1 wherein X is a halide such as iodide or bromide is subjected to a nucleophilic attack reaction with the tertiary amine 2 wherein L is the linker to give the quaternary amine 3. Other good leaving groups which can be employed in this reaction include sulfonate esters such as mesylate or tosylate. This step directly attaches the linker to the vital fluorochrome through the quaternary amine. The linker must carry a reactive group (Y) that can be used to attach the reporter group to the vital fluorochrome and linker intermediate 3. Reactive groups (Y) that are useful include an amine or carboxyl acid or ester which can undergo an amide coupling reaction. Alternatively, a reactive group such as an azide or terminal alkyne can also be used for attachment of the reporter group using “click” chemistry. The reporter group (Rp-Z) is then attached to the linker through the reactive group (Z). If amide coupling chemistry is used to attach the reporter group to the linker then Y can be either an amino group or a carboxylic acid or ester. If “click” chemistry is employed to install the reporter group onto the linker then Y can be either an azide or terminal alkyne and Z can be either an azide or terminal alkyne.

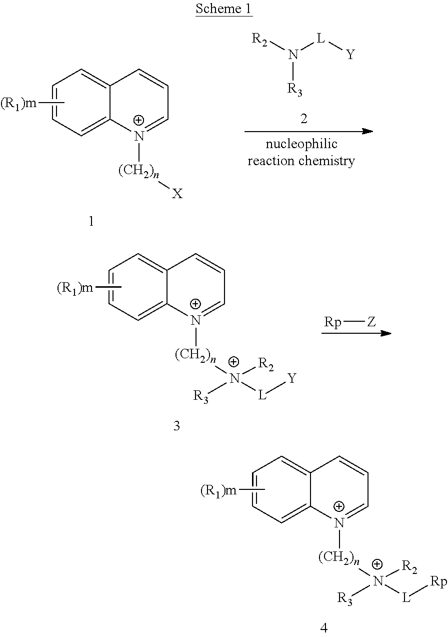

Alternatively, the linker can be initially attached to a reporter group to form amine 6, which then undergoes nucleophic attack chemistry with halide 5 to produce the conjugate 7 as shown in Scheme 2.

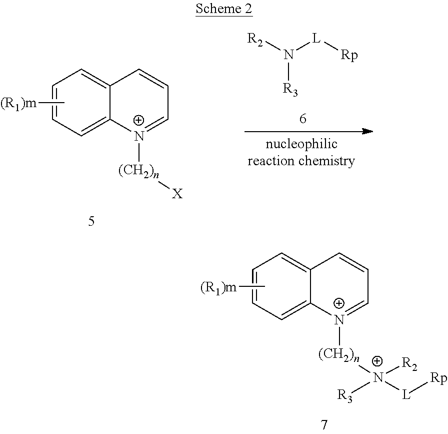

The covalent bond between the linker (L) and the reporter group (Rp) can be, but is not in any way limited to, an amide bond, an ester bond, an ether or amino bond linkage, or a triazole moiety synthesized through the reaction of a terminal alkyne and an azide via “click chemistry.”

Various methods are known to attach the reporter group to the second end of the linker using covalent bonds. In one example, the linker can be attached to the reporter group by forming an amide bond between a carboxyl (or maleimide) on the linker and the amine located on the reporter group. Reagents that can be used include EDC/NHS (N-Ethyl-N′-(3-dimethylaminopropyl)carbodiimide/N-hydroxysuccinimide) or SPDP (N-Succinimidyl 3-[2-pyridyldithio]-propionate) in both aqueous and organic solvents (such as, but not limited to, dichloromethane, acetonitrile, chloroform, tetrahydrofuran, acetone, formamide, dimethylformamide, pyridines, dioxane, or dimethysulfoxide).

In addition, various methods are known to attach the linker to the reporter group using “click chemistry” (see, e.g., the Sigma Aldrich catalog and U.S. Pat. No. 7,375,234, which are both incorporated herein by reference in their entireties). Of the reactions including “click” chemistry, one example is the Huisgen 1,3-dipolar cycloaddition of alkynes to azides to form 1,4-disubstituted-1,2,3-triazoles. The copper (I)-catalyzed reaction is mild and very efficient, requiring no protecting groups, and requiring no purification in many cases. The azide and alkyne functional groups are generally inert to biological molecules and aqueous environments.

### Methods of Detecting Vital Fluorochrome Imaging Conjugates

The vital fluorochrome conjugates of the present invention can be imaged by MR imaging (MRI), positron emission tomography (PET), single photon computerized tomography (SPECT), or other whole body imaging modalities either alone or in combination with other traditional imaging modalities such as NIR imaging. The vital fluorochrome of the vital fluorochrome conjugate can be imaged by these whole body imaging modalities to detect dead and dying cells while at the same time targeting a reporter group such as a paramagnetic metal, radiolabeled atom, or CLIO particle to the dead or dying cells. Additionally, these vital fluorochromes can be detected by traditional fluorescence imaging techniques allowing for the facile tracking of the vital fluorochrome by fluorescence microscopy or flow cytometry using methods known in the art, e.g., as described in US 2005/0249668.

### Uses of Vital Fluorochrome Imaging Conjugates

### In Vivo Imaging

The compounds and compositions described herein can be used in in vivo imaging methods to identify and evaluate cell death, injury, apoptosis, and necrosis. In general, such methods include administering to a subject one or more vital fluorochrome conjugates described herein; optionally allowing the vital fluorochrome conjugate to distribute within the subject; exposing the subject to light of a wavelength absorbable by the vital fluorophore (VF) to determine the position of the VF; and imaging the subject by either MRI, PET, SPECT, or other whole body imaging modality to detect the presence of the dead or dying cells in the subject. Furthermore, it is understood that the methods (or portions thereof) can be repeated at intervals to evaluate the subject over time.

Information provided by such in vivo imaging, for example, the presence, absence, or level of emitted signal, can be used to detect and/or monitor tissue damage, inflammation, and/or disease in the subject. Examples of causes of tissue damage include, without limitation, Alzheimer's disease, atherosclerosis, cancer, stroke, inflammatory bowel disease, diabetes, and organ transplant. In addition, in vivo imaging can be used to assess the effect of a compound or therapy by using the vital fluorochrome conjugate, wherein the subject is imaged prior to and after treatment with the compound or therapy, and the corresponding signal/images are compared. For example, a subject can be imaged prior to and after treatment with chemotherapy or radiation therapy to determine the response of the cancer cells to treatment.

The methods and compositions described herein can be used to help a physician or surgeon to identify and characterize areas of disease, such as diabetes, cancers, and atherosclerosis, and to distinguish between dead or dying tissue after suffering a heart attack or stroke. For example, vital fluorochrome conjugates can be used to help better determine when tissue damage occurred as a result of myocardial infarction (MI), which can be between 1 and 10 days, e.g., at 1 to 24 hours, at 24 to 72 hours, and over 72 hours after the MI occurred, often when critical decisions regarding treatment options are made.

The methods and compositions described herein can also be used in the detection, characterization, and/or determination of the localization of a disease, especially early disease, the severity of a disease or a disease-associated condition, the staging of a disease, and/or monitoring a disease. The presence, absence, or level of an emitted signal can be indicative of a disease state.

The methods and compositions disclosed herein can also be used to monitor and/or guide various therapeutic interventions, such as surgical procedures, and monitoring drug therapy, including cell based therapies. The methods can also be used in prognosis of a disease or disease condition.

With respect to each of the foregoing, examples of such disease or disease conditions that can be detected and/or monitored (before, during or after therapy) include inflammation (for example, inflammation caused by arthritis, for example, rheumatoid arthritis), cancer (for example, colorectal, ovarian, lung, breast, prostate, cervical, testicular, skin, brain, gastrointestinal, pancreatic, liver, kidney, bladder, stomach, leukemia, mouth, esophageal, bone), cardiovascular disease (for example, atherosclerosis and inflammatory conditions of blood vessels, ischemia, hypertension, stroke, myocardial infarction, thrombosis, disseminated intravascular coagulation), infectious disease (for example, bacterial, viral, fungal and parasitic infections, including Acquired Immunodeficiency Syndrome, Malaria, Chagas Disease, Schistosomiasis), immunologic disease (for example, an autoimmune disorder, lymphoma, multiple sclerosis, rheumatoid arthritis, diabetes mellitus, lupus erythematosis, myasthenia gravis, Graves disease), and surgery-related complications (such as organ rejection, alterations in wound healing, fibrosis or other complications related to surgical implants).

The methods and compositions described herein, therefore, can be used, for example, to determine the presence and localization of vascular disease including areas at risk for acute occlusion (i.e., vulnerable plaques) in coronary and peripheral arteries, regions of expanding aneurysms, and ischemic areas. The methods and compositions can also be used for drug delivery and to monitor drug delivery, especially when drugs or drug-like molecules are chemically attached to the imaging agents.

### In Vitro Imaging

With respect to in vitro imaging methods, the compounds and compositions described herein can be used in a variety of in vitro assays. An exemplary in vitro imaging method comprises: contacting a sample, for example, a biological sample, with one or more imaging agents of the invention; allowing the agent(s) to interact with a biological target in the sample; optionally, removing unbound agents; illuminating the sample with light of a wavelength absorbable by a fluorophore of the agents; and detecting a signal emitted from fluorophore thereby to determine whether the agent has been activated by or bound to the biological target.

After an agent has been designed, synthesized, and optionally formulated, it can be tested in vitro by one skilled in the art to assess its biological and performance characteristics. For instance, different types of cells grown in culture can be used to assess the biological and performance characteristics of the agent. Cellular uptake, binding or cellular localization of the agent can be assessed using techniques known in the art, including, for example, fluorescent microscopy, fluorescence-activated cell sorting (FACS) analysis, immunohistochemistry, immunoprecipitation, in situ hybridization and Forster resonance energy transfer (FRET) or fluorescence resonance energy transfer. By way of example, the agents can be contacted with a sample for a period of time and then washed to remove any free agents. The sample can then be viewed using an appropriate detection device such as a fluorescent microscope equipped with appropriate filters matched to the optical properties of a fluorescent agent. Fluorescence microscopy of cells in culture or scintillation counting is also a convenient means for determining whether uptake and binding has occurred. Tissues, tissue sections and other types of samples such as cytospin samples can also be used in a similar manner to assess the biological and performance characteristics of the agents. Other detection methods including, but not limited to flow cytometry, immunoassays, hybridization assays, and microarray analysis can also be used.

### Compositions

The vital fluorochrome conjugates described herein can be provided dry or dissolved in a carrier or vehicle, e.g., pharmaceutically acceptable carriers and vehicles. Useful carriers and vehicles include, but are not limited to, buffer substances such as phosphate, glycine, sorbic acid, potassium sorbate, tris(hydroxymethyl)amino methane (“TRIS”), partial glyceride mixtures of fatty acids, water, salts or electrolytes, disodium hydrogen phosphate, potassium hydrogen phosphate, and sodium chloride.

The vital fluorochrome conjugates can be administered in the form of a sterile injectable preparation. The possible vehicles or solvents that can be used to make injectable preparations include water, Ringer's solution, and isotonic sodium chloride solution, and 5% D-glucose solution (D5W). In addition, oils such as mono- or di-glycerides and fatty acids such as oleic acid and its derivatives can be used. The compounds and compositions can be administered orally, parenterally, by inhalation, topically, rectally, nasally, buccally, vaginally, or via an implanted reservoir. The term “parenteral administration” includes intravenous, intramuscular, intra-articular, intrasynovial, intrasternal, intrathecal, intraperitoneal, intracisternal, intrahepatic, intralesional, and intracranial injection or infusion techniques. The optical sensor conjugates can also be administered via catheters or through a needle to any tissue.

Dosing of the optical sensor conjugate will depend on a number of factors including the sensitivity of the detection system used, as well as a number of subject-related variables, including animal species, age, body weight, mode of administration, sex, diet, time of administration, and rate of excretion.

Prior to use of the invention or any pharmaceutical composition of the invention, the subject can be treated with an agent or regimen to enhance the imaging process. For example, a subject can be put on a special diet prior to imaging to reduce any auto-fluorescence or interference from ingested food, such as a low pheophorbide diet to reduce interference from fluorescent pheophorbides that are derived from some foods, such as green vegetables. Alternatively, a cleansing regimen can be used prior to imaging, such as those cleansing regimens that are used prior to colonoscopies and include use of agents such as Visiciol™. The subject (patient or animal) can also be treated with pharmacological modifiers to improve image quality. For example, using low dose enzymatic inhibitors to decrease background signal relative to target signal (secondary to proportionally lowering enzymatic activity of already low-enzymatic activity normal tissues to a greater extent than enzymatically-active pathological tissues) can improve the target-to-background ratio during disease screening.

### EXAMPLES

The following examples are illustrative and not limiting.

### Example A

### Determining the Properties of Vital Fluorochrome Conjugates

Experimental Details of the Relaxation Assay: Gadolinium thiazole orange (GadoTO) at 1 mM was employed with increasing concentrations of plasmid DNA (DNA 0.025 to 0.3 ug/uL). Relaxation times were measured on a Bruker minispec at 20 mHz. T1 values were fit to a non-linear sigmoidal dose-response regression, GraphPad® Prism) to determine the half-maximal concentration (EC50=0.089 mM, 95% confidence interval 0.078-0.101).

Experimental Details of the Fluorescence Assay: GadoTO at 2.35 uM was employed with increasing concentrations of DNA. Absorption spectra were measured on a Varian Cary 50 Bio UV-Visible spectrophotometer. The absorbance at 511 nm was measured. Fluorescence emission spectra were recorded on a Varian Cary Eclipse fluorescence spectrophometer. Fluorescence intensities were measured between 510 and 700 nm and corrected, when necessary, for matched absorbances at 511 nm. Fluorescence intensities were plotted versus DNA concentrations. Fluorescence values were fit to a non-linear sigmoidal dose-response regression (GraphPad Prism) to determine the half-maximal concentration (EC50=0.179 mM, 95% confidence interval 0.149-0.214).

Results from the Relaxation and Fluorescence Assays: The design of vital fluorochrome conjugates must preserve the essential features of the vital fluorochrome upon which it is based. The verification that a putative vital fluorochrome conjugates (VF-Linker-Reporter) has the properties of the VF upon which its was based is shown in FIGS. 2A, 2B, 3 and 4 which show the results with the vital fluorochrome conjugates known as GadoTO. The binding of GadoTO to a double stranded plasmid DNA is shown in FIG. 2A and FIG. 2B. Since the reporter group of GadoTO is a paramagnetic gadolinium chelate, binding can be ascertained by changes in the water relaxation rates. FIG. 2A shows the binding as a decrease in T1 (the spin-spin relaxation of water) of a solution of GadoTO with increasing concentrations of DNA. Binding of small GadoTO to the larger DNA slows the molecular motion, increasing the R1 relaxivity and decreasing the solution T1. The binding of GadoTO to DNA can also be ascertained by an increase in fluorescence as shown in FIG. 2B.

Experimental Details for the Dual Wavelength FACS and Single Channel FACS: Cell culture: All experiments described used Jurkat T cells (Clone E6-1, ATCC #TIB-152). Cells were grown in RPMI 1640 medium (ThermoFisher Scientific, Hampton, N.H.) with 10% fetal bovine serum (Valley Biomedical, Winchester, Va.), 1% L-Glutamine (Mediatech, Manassas, Va.), 1% Penycillin-Streptomycin (Mediatech, Manassas, Va.). Media was changed every two or three days. Cells were maintained at 37° C., 5% CO₂. Flow cytometry experiments: Cells were treated with camptothecin (CPT, 24 hrs, 10 μM). After removal of the medium, cells were resuspended into 100 μL of binding buffer (10 mM HEPES, 140 mM NaCl, 2.5 mM CaCl₂, 1% FBS). Cells were stained at 37° C. for 15 min with 1 μL Annexin-APC (Invitrogen) and 1 μL propidium iodide (BD Pharmingen) or 1 μL of GadoTO (0.1 mM stock solution in binding buffer). Cells were diluted with 200 μL of binding buffer and analyzed by flow cytometry (FACSCalibur, Becton Dickinson). Fluorescence from the APC fluorochrome was measured in FL4, PI in FL3, and GadoTO in FL1.

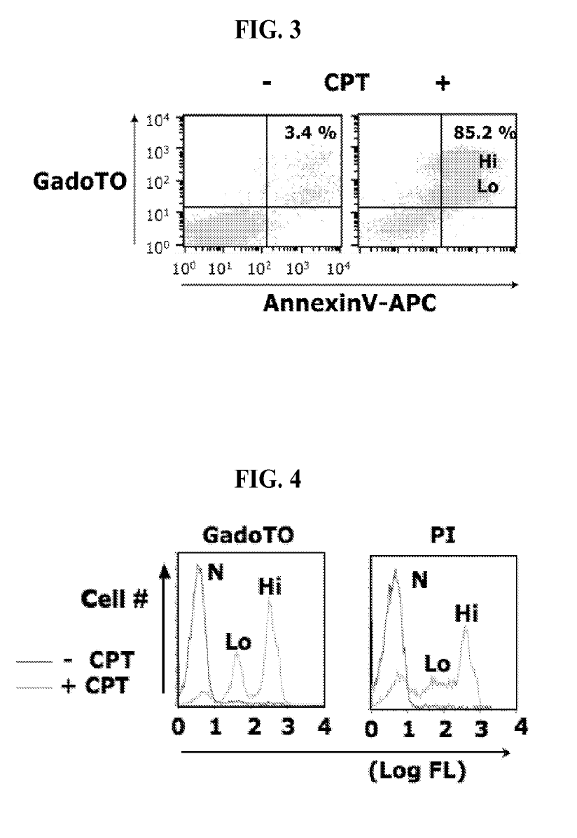

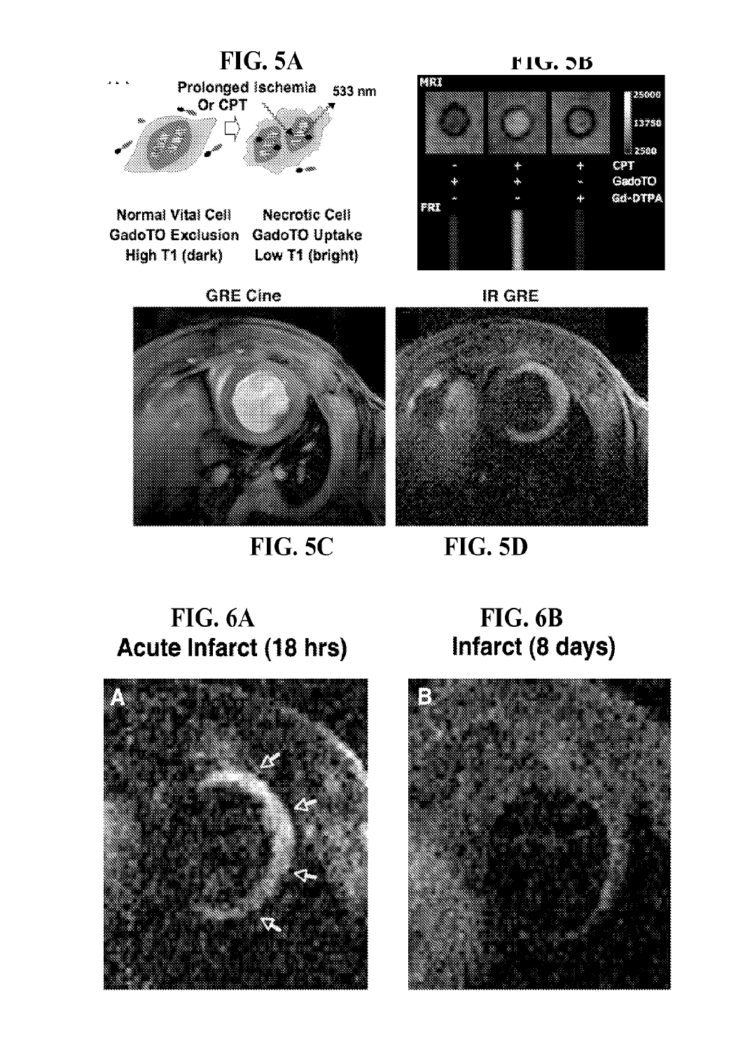

Results from Dual Wavelength FACS: The exclusion of GadoTO from healthy cells and uptake by necrotic cells by FACS is shown in FIG. 3. The uptake of GadoTO was demonstrated by culturing Jurkat T cells, inducing apoptosis and necrosis with camptothecin (CPT), and analyzing the binding of GadoTO and Annexin V-APV by dual wavelength FACS. Before camptothecin (−CPT, left panel) most cells were viable and failed to bind either GadoTO or Annexin V-APC (lower left quadrant). After CPT (+CPT, right panel), cells bound both probes (upper right quadrant), indicating they were necrotic. GadoTO binding necrotic cells were of two intensities (Hi and Lo), indicating two subpopulations of necrotic cells.

Results from Single Channel FACS: To compare the binding of GadoTO and the binding of a standard VF propidium iodide, single channel FACS was employed as shown in FIG. 4. +CPT and —CPT Jurkat cells were obtained as in FIG. 3, but cells were then treated either with GadoTO or with the reference VF, propidium iodide (PI). Without CPT (−CPT), cells failed to bind GadoTO, showed by a prominent peak with a fluorescence intensity between Log 0 and Log 1 labeled “N” (black line). With CPT, those normal “N” cells decreased and two peaks (Lo and Hi) of highly fluorescent cells were seen (gray line). Similar behavior was seen when propidium iodide (PI) replaced GadoTO.

### Example B

### Imaging Necrotic Cells In Vitro and In Vivo

The ability to image necrotic cells in vitro and in vivo with GadoTO by MRI is shown in FIG. 5A-5D. The interaction of GadoTO with necrotic cells is shown schematically in FIG. 5A. GadoTO is excluded by vital cells. Prolonged ischemia or a 48 hour exposure to CPT induces necrosis, which causes GadoTO to enter cells and intercalate with double stranded nuclear DNA. This results in (i) nuclear cell fluorescence (emission @ 533 nm), (ii) a drop in cellular T1 and, (iii) a brightening of cells on T1 weighted MR images. For in vitro studies, we exposed Jurkat cells to CPT and GadoTO, and then obtained the T1 weighted MRI image shown in FIG. 5B. CPT treated cells took up GadoTO, and were brighter than non-CPT treated cells by MRI and fluorescence reflectance imaging (FRI). Effects on cellular T1 were confirmed by relaxometry studies using cell suspensions. CPT treatment did not induce the uptake of the non-DNA binding Gd-DTPA chelate by MRI (FIG. 5B) or by relaxometry (data not shown). These results demonstrate the essential role of the VF moiety in the behavior of GadoTO.

For in vivo MR studies, we employed a model of prolonged ischemia, obtained when the left coronary artery of a beating mouse heart was ligated for 18 hours. The ligation and procedure is similar to our previously described method (Sosnovik, D. E. et al., Magn. Reson. Med., 54:718-24, 2005) except the duration is for 18 hours. This produced a predominantly necrotic response in cardiomyoctyes but did not block GadoTO perfusion of the insult due to collateral supply mechanisms. GadoTO was injected (18 hours post ligation) and imaged (21 h). A GRE image (FIG. 5C) showed anatomical detail and a barely visible infarct. However, the IR GRE image (FIG. 5D), optimized for the detection of GadoTO, showed a well-defined, bright infarct. Gd-DTPA washed in and out of damaged myocardium within 1 hour. Control images (no infract or no GadoTO showed the specificity of the agent. Thus, GadoTO was detectable by MRI in vitro (Jurkat cell/CPT induced necrosis) and by MRI in vivo (cardiomyocyte/ischemia induced necrosis).

### Example C

### Imaging Method to Determine the Age of a Myocardial Infarction

An additional feature of the invention is an imaging method to determine the age of a myocardial infarction (MI), i.e., the time since the occlusion began. Patients can present with symptoms of variable intensity and duration that may or may indicate an infarction. Current techniques can define regions of non-functional myocardium, but cannot define the time of infarction. With developing (“young”) infarctions, treatments to limit or reverse the affected area are indicated, while with “old” infarctions such treatments are futile. In this method a vital fluorochrome conjugate is injected intravenously and MRI or PET or SPECT are used to measure the vital fluorochrome conjugates uptake in an infracted area. As shown in FIG. 3, the GadoTO (synthesis from Example 1) can be used to image cell death brought about by ischemia (tying off a blood vessel) with the mouse myocardium.

The technique for determining the age of a myocardial infarction (MI) divides the post-infarction time response into four phases:

Phase 1 (Pre-infarction phase, pre MI): With oxygenated, viable cardiomyocytes vital fluorochrome conjugates are not taken up.

Phase 2 (1-24 hours post MI): Necrotic cells accumulate progressively due to oxygen deprivation. Membrane function is compromised and vital fluorochrome conjugates are retained due to binding to DNA.

Phase 3 (24-72 hours post MI): Necrosis leads to inflammation and macrophage infiltration. Degradation of DNA occurs, reducing vital fluorochrome conjugates accumulation during this phase. Vital fluorochrome conjugate still accumulates, indicating the MI within 72 hours.

Phase 4 (>72 hours post MI): Inflammatory cells have cleared DNA and vital fluorochrome conjugate binding is zero. However, the infracted area is not functional (contractile).

FIGS. 6A and 6B show an example of a beating mouse heart injected with GadoTO imaged during phase 2 (18 hours) (FIG. 6A) and phase 4 (8 days) (FIG. 6B) of the post MI reaction phases. Profound uptake of GadoTO is seen (arrows pointing to area of uptake) in the infarcted myocardium reflecting uptake of the agent in acutely necrotic tissue (FIG. 6A), while no uptake of the agent is seen in an infarcted mouse injected with GadoTO 8 days after myocardial infarction (FIG. 6B). GadoTO can thus be used to specifically image acutely necrotic myocardium/tissue.

### Example D

### Imaging Necrotic Cells and Determining Age of MI in Humans

The basic procedures outlined in Examples B and C can be repeated on a human with appropriate modifications. For example, a vital fluorochrome conjugate possessing a paramagnetic Gd chelate reporter is injected at between 0.01 to 0.2 mmoles Gd, but preferably at about 0.05 moles. Exemplary compounds are any of the MR detectable vital fluorochrome conjugates from the examples summarized in Table 4. In some cases a vital fluorochrome conjugate can be used for other applications other than diagnostic imaging.

Cell separation: Vital fluorochrome conjugates with superparamagnetic nanoparticle reporters (Examples 6 and 7, Table 4) can be used for the removal of necrotic cells from vital cells in cell mixtures using magnetic separators. Here necrotic cells are selectively magnetized and removed with a magnetic separator. Magnetic-annexin V's, which bind apoptotic and necrotic cells, are employed for the removal of those cells and sold by MBL-International, BioVision, and Miltenyi Biotech.

TABLE 4
Summary of Examples

| Ex. | Vital Fluorochrome Conjugate | Comment |
|---|---|---|
| 1 | 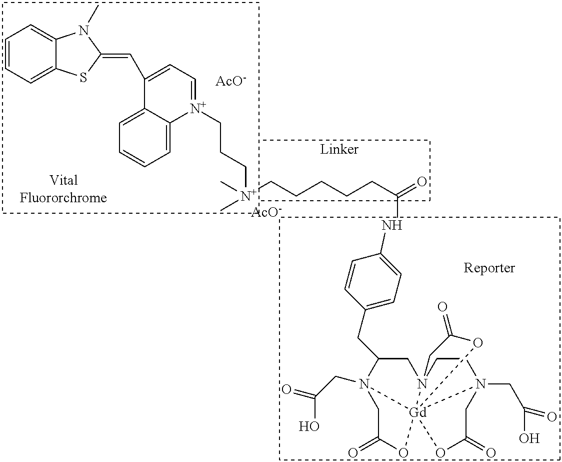 | GadoTO, MRI detection. Vital fluorochrome, linker and Gd-chelate reporter group are shown |
| 2 | 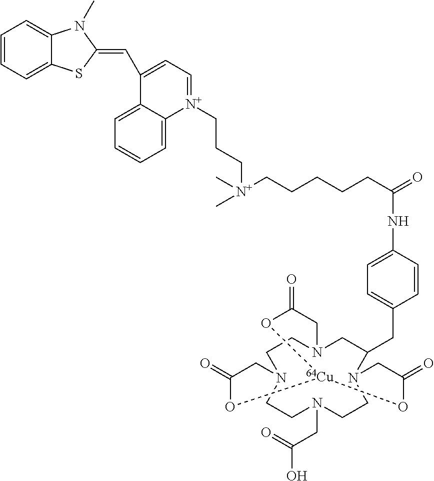 | PET detection |
| 3 | 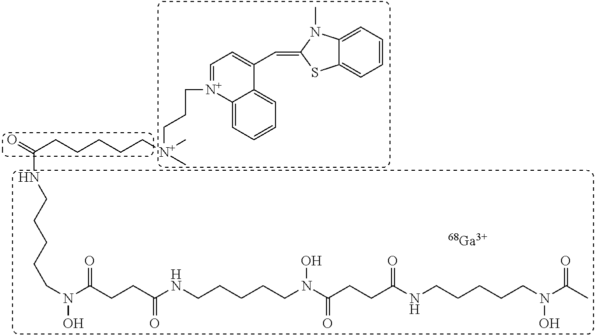 | PET detection Reporter group is deferoxamine with a ⁶⁸Ga³⁺ atom. |
| 4 | 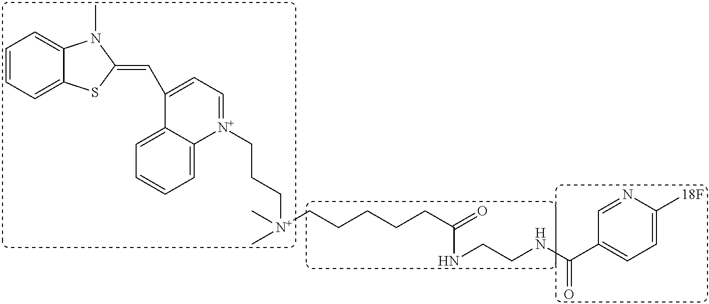 | PET Detection |
| 5 | 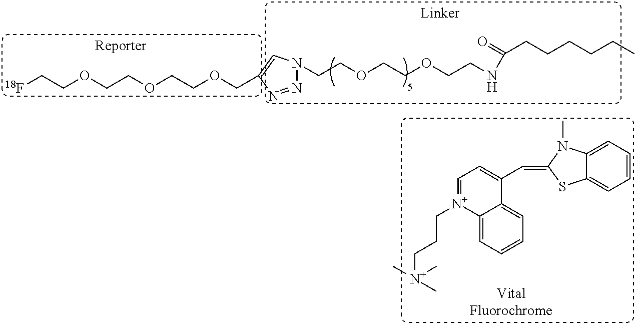 | Use of PEG in linker and click chemistry. |
| 6 | 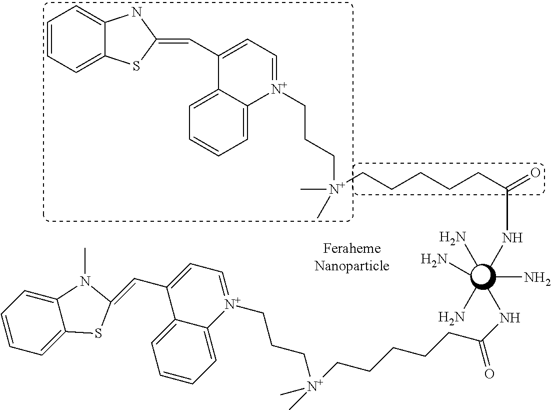 | Reporter group = polymer coated super- paramagnetic iron oxide nanoparticle detected by MRI |
| 7 | | See example 6. |
| 8 | 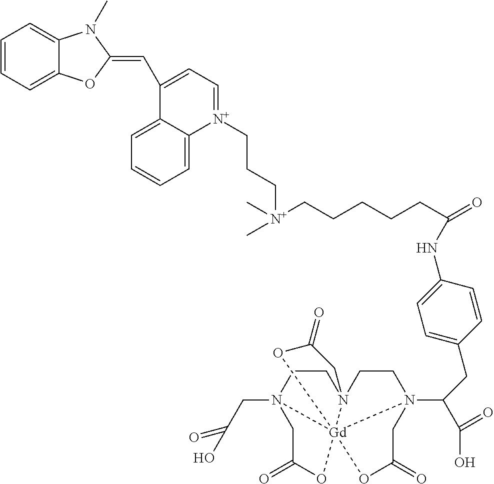 | YO replaces TO in example 1 MR detection |
| 9 | 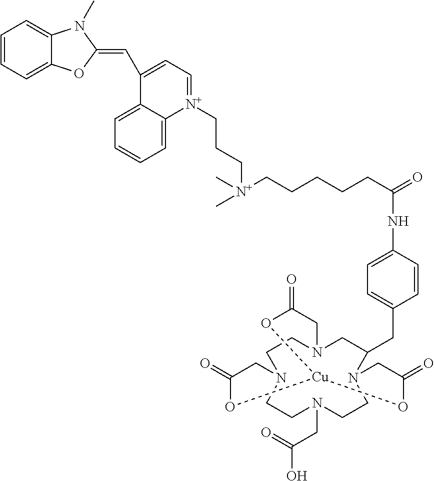 | YO replaces TO from example 2, MR detection |
| 10 | 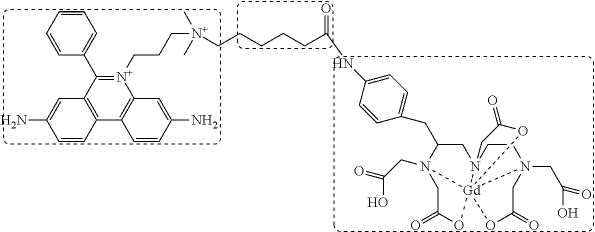 | Propidium based vital fluorochrome conjugate, MR detection |

### Example 1

### Synthesis of GadoTO

The synthesis and use of gadolinium thiazole orange (GadoTO) (Ex. 1 in Table 4) has been previously described (Garanger, E. et al. “A DNA-binding Gd chelate for the detection of cell death by MRI” Chemical communications 4444-6, 2009).

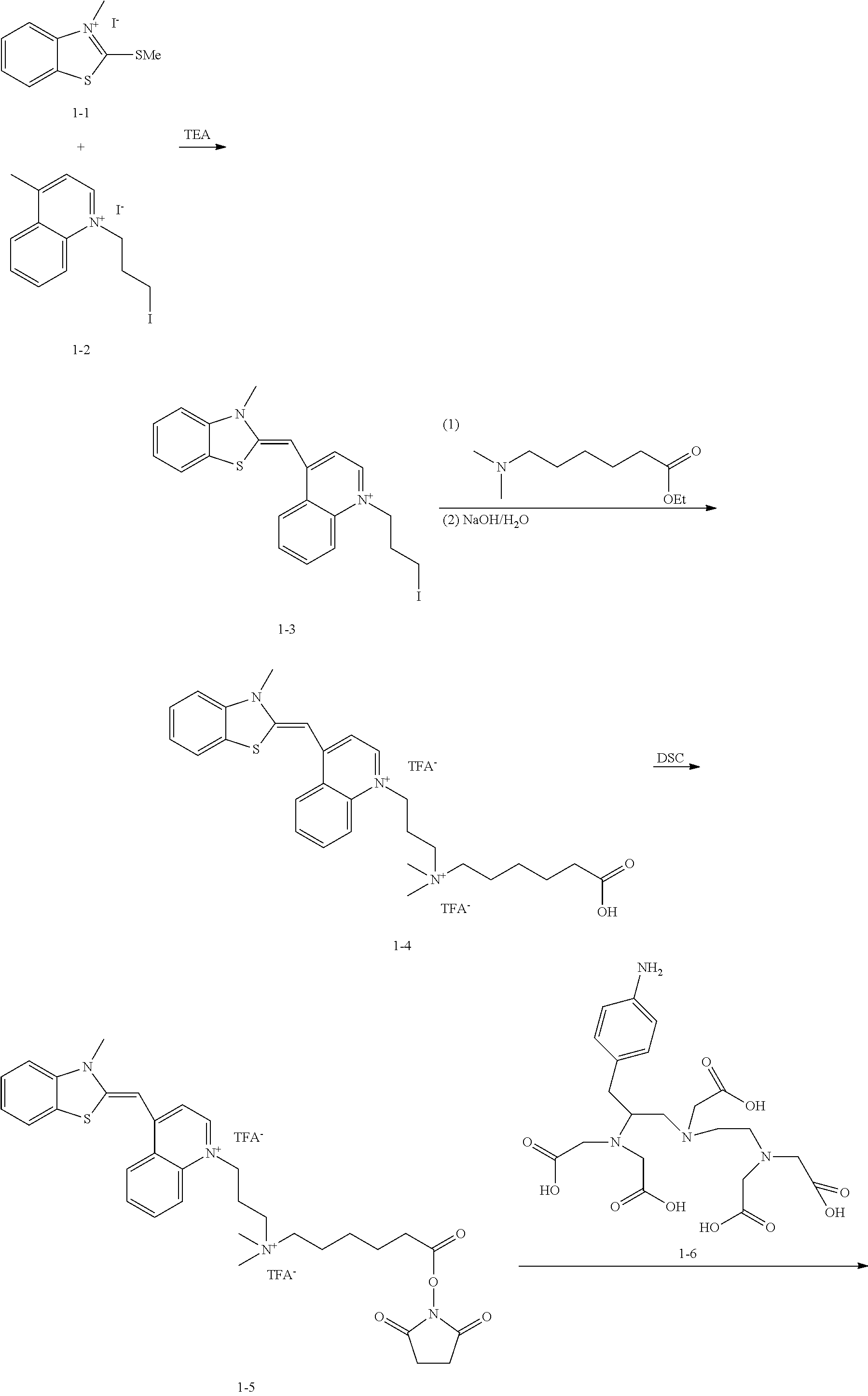

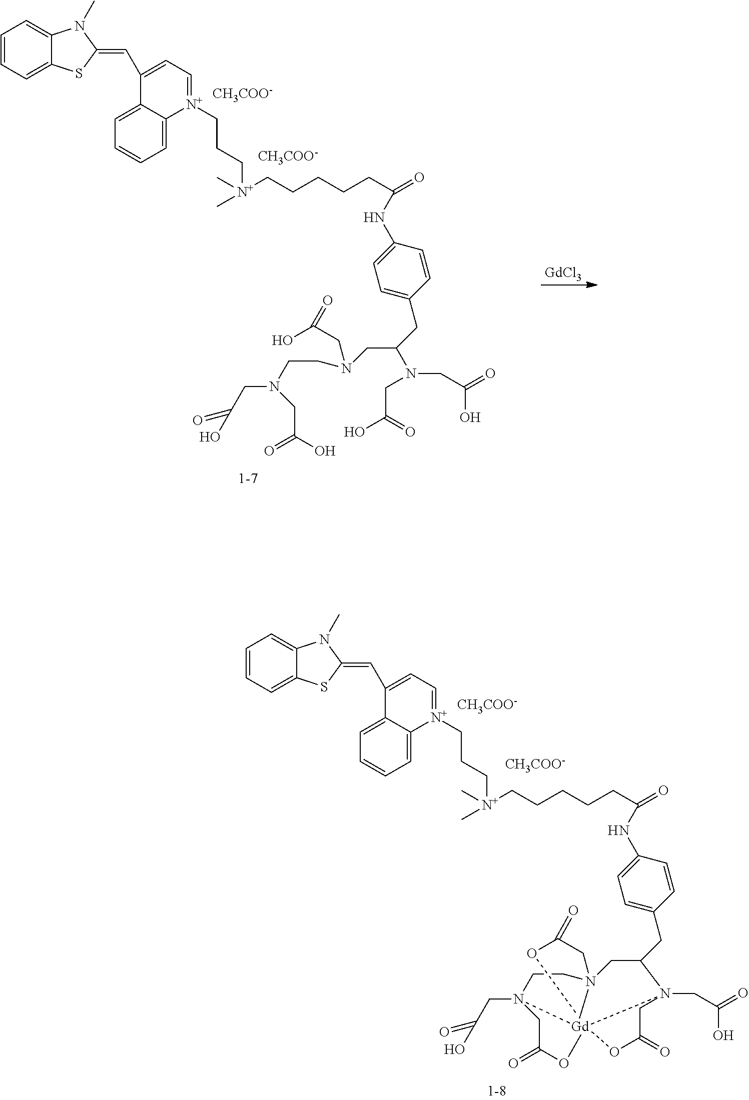

### Synthesis of TO-Iodide 1-3

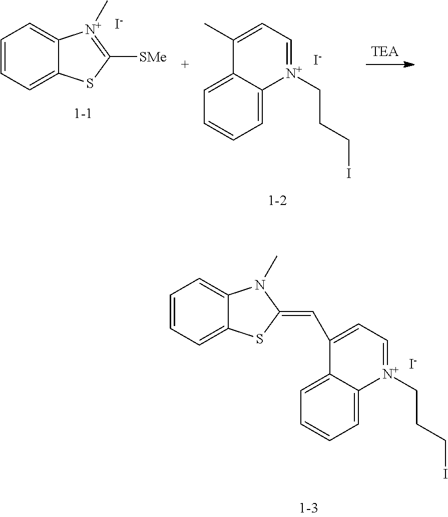

To a sealed tube (48 ml size) were added thioazolium (1-1) (500 mg, 1.55 mmol) and quinolinium (1-2) (679 mg, 1.55 mmol) in methanol (2 ml, as little as possible). With a heating gun the tube was heated until the solid dissolved. Heating was stopped and the cap was opened carefully. After triethylamine was added (259 μl, 1.2 eq), the reaction mixture turned dark red immediately. The flask was shaken and MeSH generated by the reaction was released by opening the mouth of the tube. The jelly-like solid was stirred for 1 hour at room temperature. A dark red solid was obtained with ether precipitation. The solid was suspended in methanol (2 ml) and re-precipitated with ether (40 ml). The solid was collected by centrifuge in a falcon conic tube. The solid was re-suspended in methanol (2 ml) and sonicated for 2 minutes. The solution was centrifuged and the dark solution was discarded. The solid was washed with ether again (40 ml) and dried under vacuum. The red solid was characterized by LCMS: 459. A high purity sample was obtained by HPLC purification. The presence of the by-product of TO-SMe was removed by HPLC purification in the next step.

### Synthesis of TO-Acid 1-4

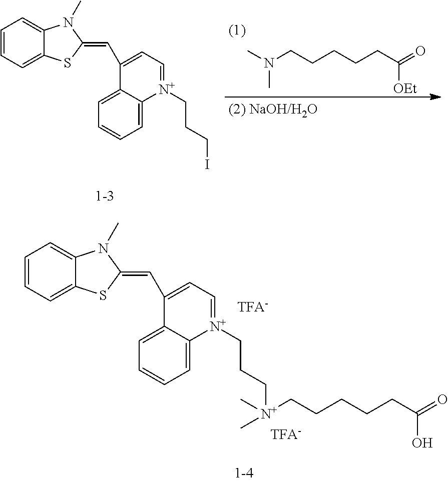

In a sealed tube, l′-(3′-iodopropyl)-3-methyl-oxa-4′-cyanine iodide 1-3 (200 mg, 341.1 mmol) was suspended and dissolved into 10 mL anhydrous ethanol. A solution of ethyl N,N-dimethyl-6-aminohexanoate (320 mg, 1.709 mmol, 5 equiv.) in 2 mL anhydrous ethanol was added. The reaction mixture was stirred overnight at 145° C. The dark purple solution was cooled to room temperature. The precipitate formed was separated by filtration, washed with ether and dried under vacuum. The purple solid powder was dissolved in 8 mL of a mixture of acetonitrile/water (50:50). A solution of NaOH 1M (4 mL) was added. The reaction mixture was stirred at room temperature for 1 hour and purified by RP-HPLC (UV monitoring at 500 nm, 0-100% eluant B in 30 minute gradient, eluant A: 0.1% TFA, eluant B: 0.1% TFA, 90% acetonitrile, 21 mL/minute flow rate). The fraction collected (Rt=13 minutes) was lyophilized.

Compound 1-4 was obtained as an orange solid powder (94.5 mg, 156.5 mmol, 45.9% yield). (C29H36N3O2S+, CF₃CO₂⁻). Calc. exact mass: 490.25+112.99; found m/z: [M+H]2+=245.7. 1H NMR (400 MHz, DMSO-d6, ppm): 1.28 (quintet, 2H, J=8 Hz); 1.56 (quintet, 2H, J=8 Hz); 1.69 (m, 2H); 2.30 (m, 4H); 3.05 (s, 6H); 3.29 (m, 2H); 3.50 (m, 2H); 4.07 (s, 3H); 4.65 (t, 2H, J=8 Hz); 7.0 (s, 1H); 7.41 (d, 1H, J=8 Hz); 7.46 (t, 1H, J=8 Hz); 7.65 (t, 1H, J=8 Hz); 7.79 (t, 1H, J=8 Hz); 7.84 (d, 1H, J=8 Hz); 8.03 (t, 1H, J=8 Hz); 8.10 (d, 1H, J=8 Hz); 8.22 (d, 1H, J=8 Hz); 8.65 (d, 1H, J=8 Hz); 8.85 (d, 1H, J=8 Hz).

### Synthesis of TO Acid NHS Ester 1-5

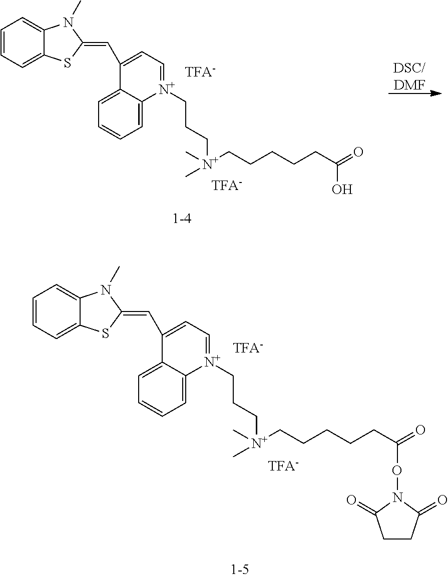

Compound 1-4 (94.5 mg, 156.5 μmol) was dissolved in 1.3 mL anhydrous DMF. Anhydrous DIPEA (70 μL, 401.9 μmol, 2.6 equiv.) and di(N-succinimidyl) carbonate (101.2 mg, 395.1 μmol, 2.5 equiv.) were added. The reaction mixture was stirred at room temperature for 1 hour. Precipitation from ether afforded the succinimidyl ester intermediate as a red solid.

### Synthesis of TO-DTPA 1-7

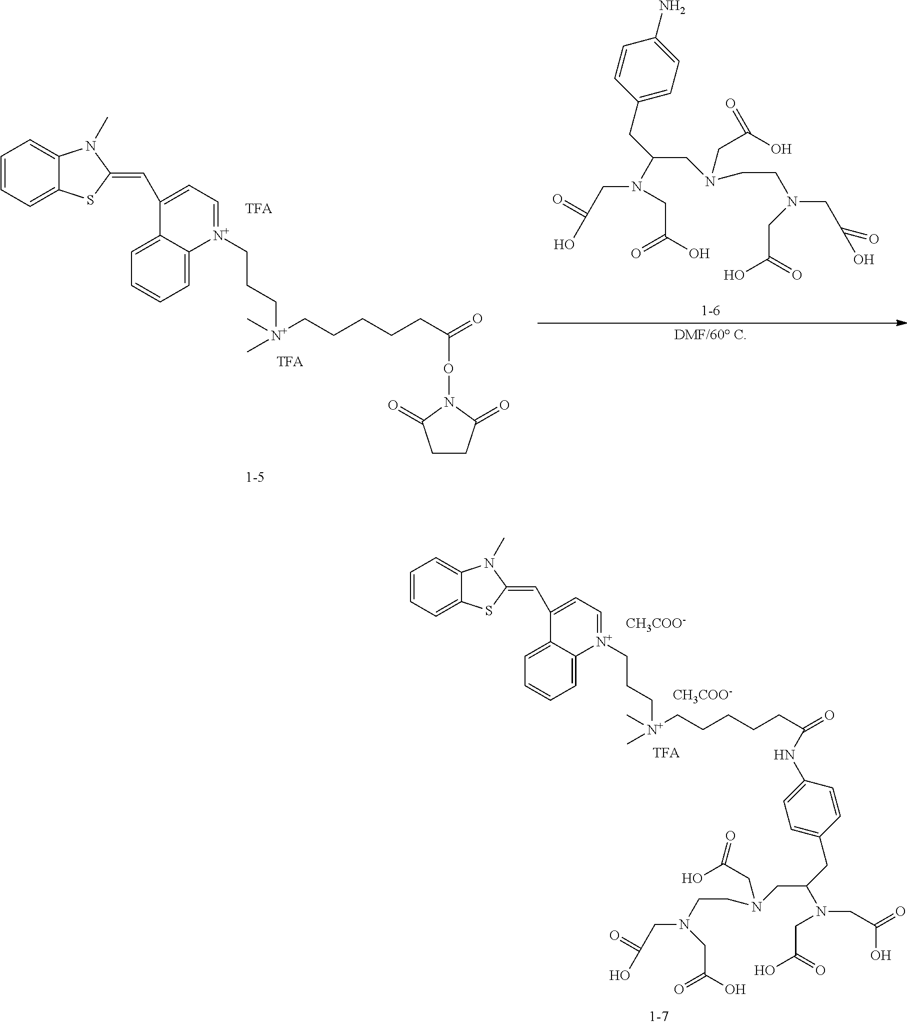

In a polypropylene vial, p-NH2-Bn-DTPA 1-6 (70.4 mg, 141.2 μmol) was dissolved in 706 μL anhydrous DMF containing DIPEA (295 μL, 1.694 mmol, 12.0 equiv.). A solution of the succinimidyl ester intermediate 1-5 (78.3 umol, 0.55 equiv.) in 330 μL anhydrous DMF was added. The reaction mixture was stirred overnight at 60° C. and purified by RP-HPLC (UV monitoring at 500 nm; 0-50% eluant B in 30 minute gradient; eluant A: ammonium acetate buffer (50 mM); eluant B: acetonitrile; 21 mL/minute flow rate). The fraction collected (Rt=15 min) was lyophilized. Compound 1-7 was obtained as a red solid (31.4 mg, 32.4 μmol, 20.7% yield). (C50H63N7O11S). Calc. exact mass: 969.43; found m/z: [M+2H]2+=486.0, [M+3H]3+=324.2.

### Synthesis of GadoTO 1-8

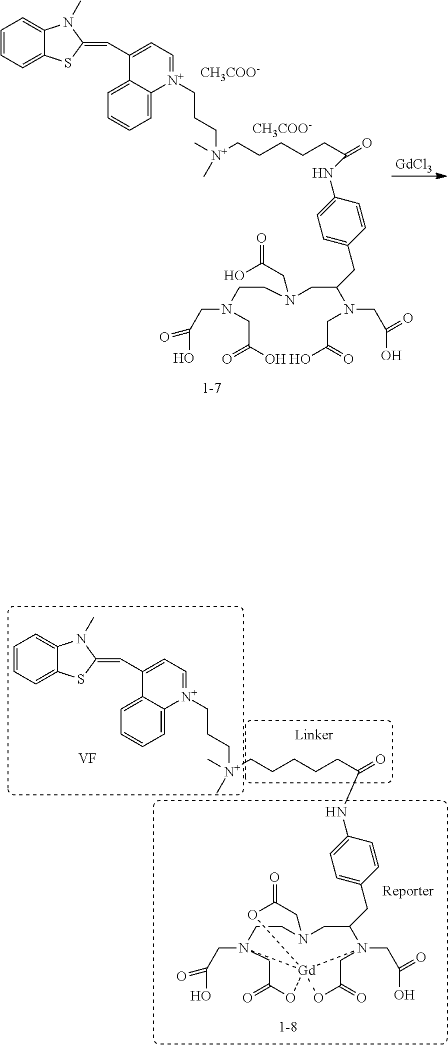

Compound 1-7 (23.8 mg, 24.5 μmol) was dissolved in 900 μL of a solution of citric acid 0.1 M pH 5.7. GdCl₃.6H₂O (37.0 mg, 99.5 umol, 4.1 equiv.) dissolved in 1.8 mL citric acid 0.1 M pH 5.7 was added. The reaction mixture was stirred for 2 hours at room temperature and purified by RP-HPLC (UV monitoring at 500 nm; 0-50% eluant B in 30 minute gradient; eluant A: ammonium acetate buffer 50 mM; eluant B: acetonitrile; 21 mL/minute flow rate). The fraction collected (Rt=17 min) was lyophilized. Compound 1-8 (GadoTO) (27.2 mg, 24.2 umol, quant. yield) was obtained as an orange solid powder. (C50H60GdN7O11S). Calc. exact mass: 1124.33; found m/z: [M+2H]2+=563.5.

### Example 2

### Synthesis of ⁶⁴Cu-DOTA-TO and ⁶⁸Ga-DOTA-TO

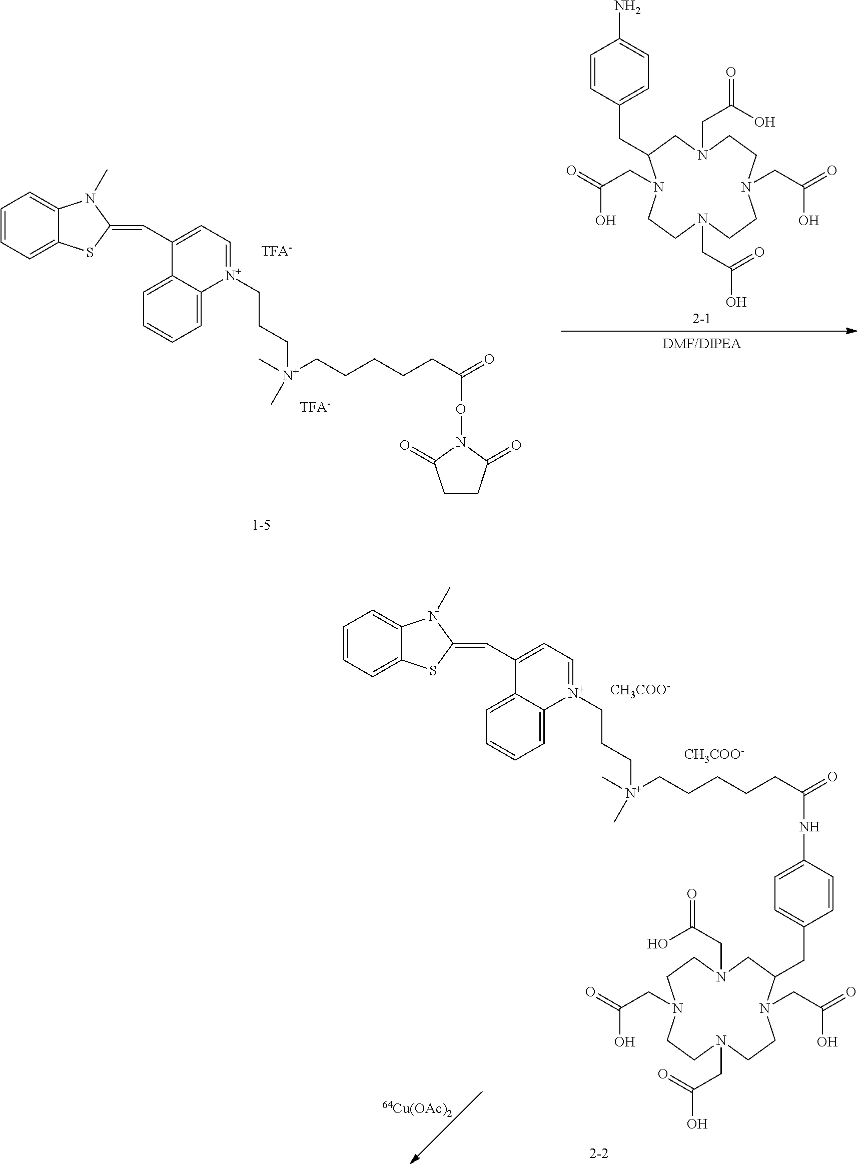

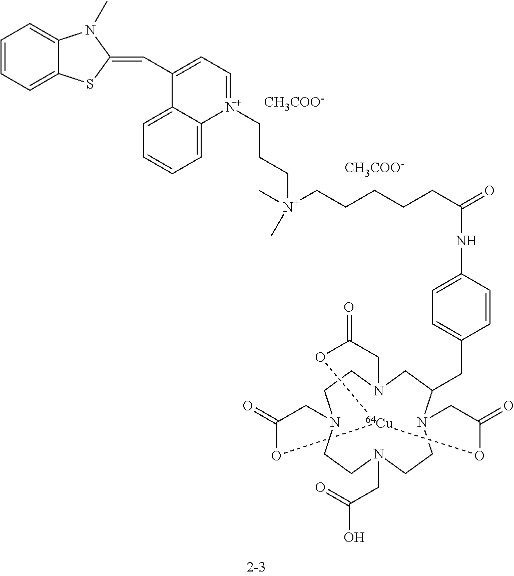

### Synthesis of DOTA-TO 2-2

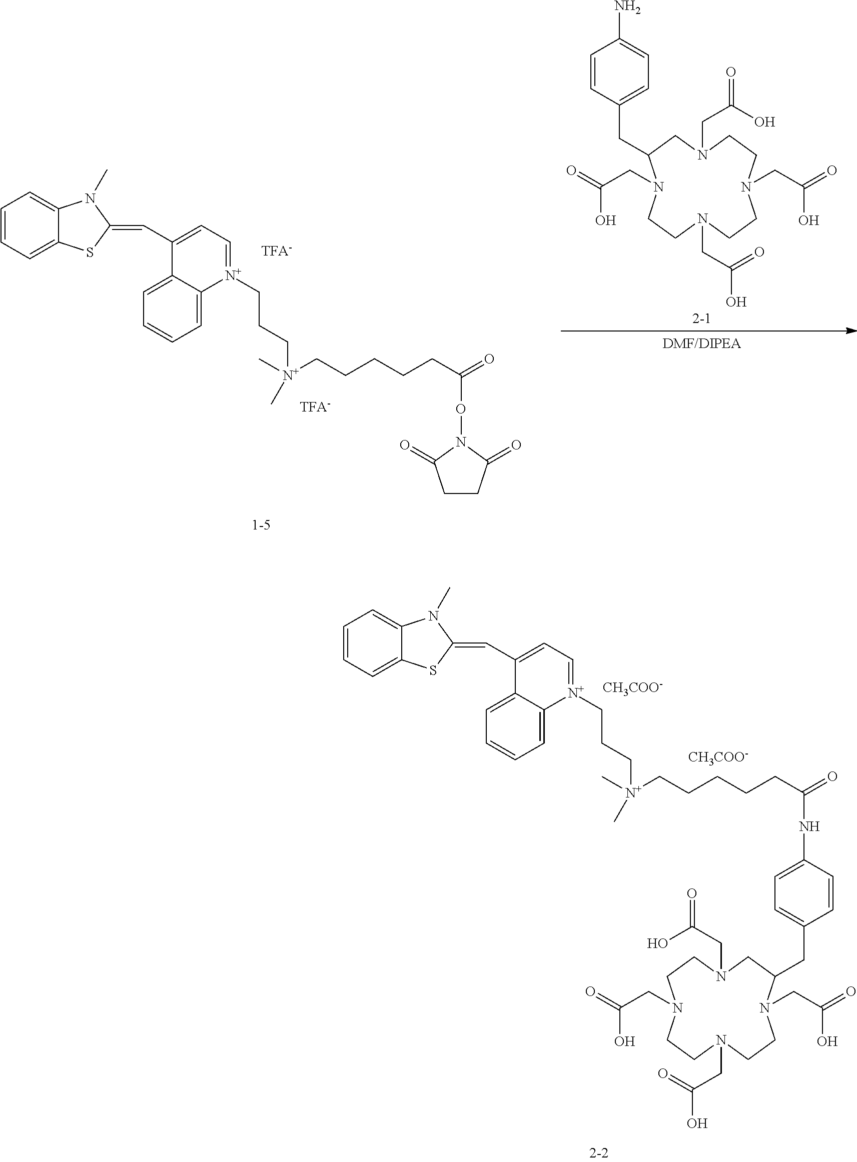

### Synthesis of DOTA-TO 2-2

TO-Acid NHS ester 1-5 (25.8 mg, 25.8/814.8=31.7 mmol) was dissolved in DMF (600 ul). A solution of p-NH₂BnDOTA (32.3 mg, 32.3/509.6=63.4 mmol, 2 eq) in anhydrous DMF (400 ul) and DiPEA (132.5 ul, 12 eq to 1 DOTA, 0.761 mmol) was added to the solution of NHS eater. The solution was stirred under 60° C. overnight. RP-C18 HPLC was used for the purification (Buffer A: NH₄OAc 50 mM, Buffer B: acetonitrile) at 500 nm, flow: 10 ml/minute 0-50% B in 20 minutes, back to 10% B in 5 minutes and isocratic for 5 minutes. Product was collected at 16.83 minutes and confirmed by MS: 491 (M²⁺). The fractions were lyophilized until dry. 8.5 mg, yield: 24%.

### Synthesis of ⁶⁴Cu-DOTA-TO 2-3a

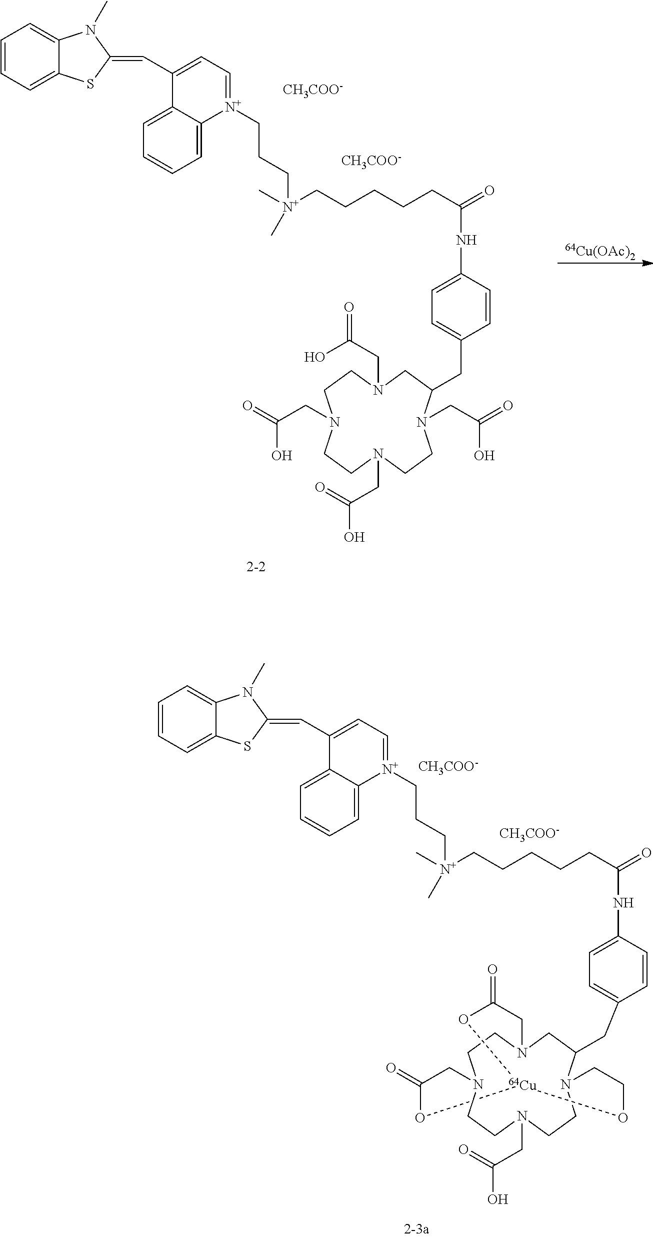

To DOTA-TO 2-2 (55 ug, 0.05 μmol) labeling with metal chelation is carried out with ⁶⁴Cu²⁺ by addition of 2 mCi of ⁶⁴Cu²⁺ in 0.1 M sodium acetate buffer (pH 5.5) followed by 45 minutes of incubation at 45° C. The radiochemical yield is determined by radio-TLC at different time points. Radio-TLC is performed with MKC18 silica gel 60-Å plates (Whatman, New Jersey, USA) with solvent MeOH/20% NaOAc as the eluent using a Bioscan AR2000 imaging scanner (Bioscan, Washington, D.C., USA) and Winscan 2.2 software. ⁶⁴Cu-DOTA-TO is purified by radio-HPLC performed on a C18 reversed phase column (5 um, 250×4.6 mm) and monitored using a radiodetector and a UV detector at 500 nm. The flow rate is 1.0 ml/minute, with the mobile phase solvent A (0.1% TFA in water) and solvent B (0.1% TFA in acetonitrile). The eluant is evaporated and reconstituted in saline, which is filtered into a sterile dose vial for use in animal experiments by passage through a 0.22-μm Millipore filter.

### Synthesis of 68Ga-DOTA-TO 2-3b

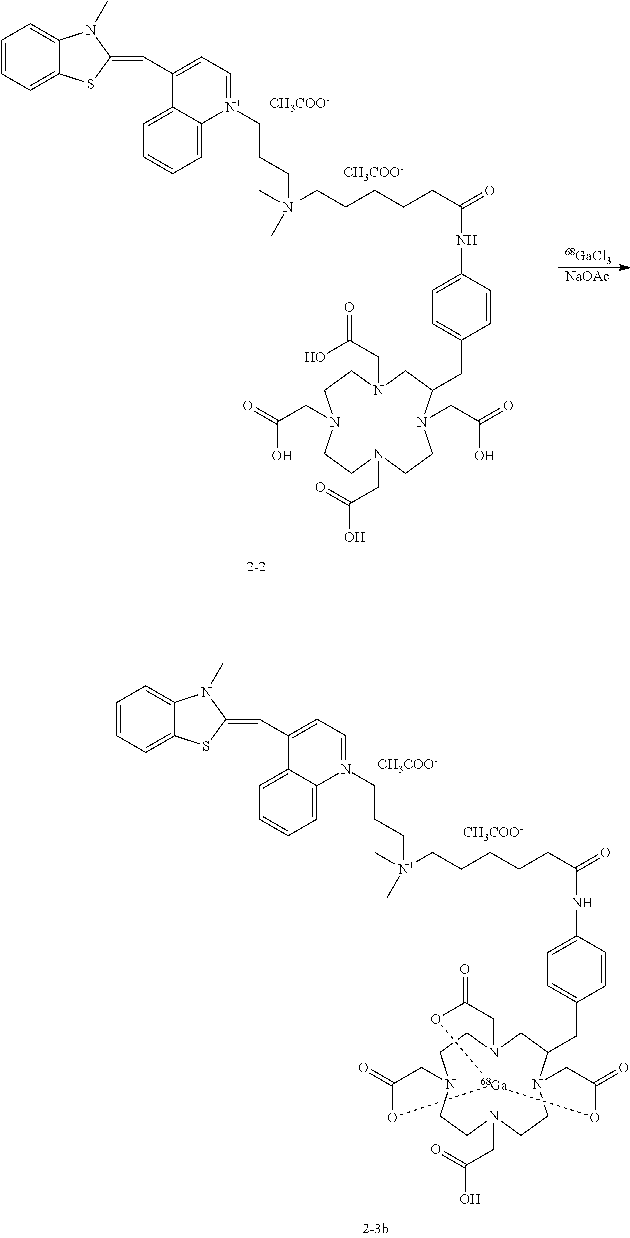

The ⁶⁸Ge—⁶⁸Ga generator is used to generate ⁶⁸Ga. Elution is performed with 0.1 M ultrapure HCl (prepared from ultrapure HCl 30% TraceSelectUltra and Ultrapure water, Fluka, Buchs, Switzerland). Labeling is performed by adding 10 μg DOTA-TO and 8 μl 2.5 M Na-acetate to 150 ul eluate, containing 8-10 MBq ⁶⁸Ga activity, and by heating the reaction solution for 10 minutes at 80° C. The reaction solution is then cooled in ice-cold water for 5 minutes; 5 μl EDTA 5 mM is then added to chelate any residual ⁶⁸Ga. Saline is added to reach a final volume of 600 ul, which is used for two animals, each receiving 5 μg TO and ca. 3.7 MBq ⁶⁸Ga. Radiopharmaceutical purity is assessed by high-performance liquid chromatography (HPLC).

### Example 3

### Synthesis of Deferoxamine-TO

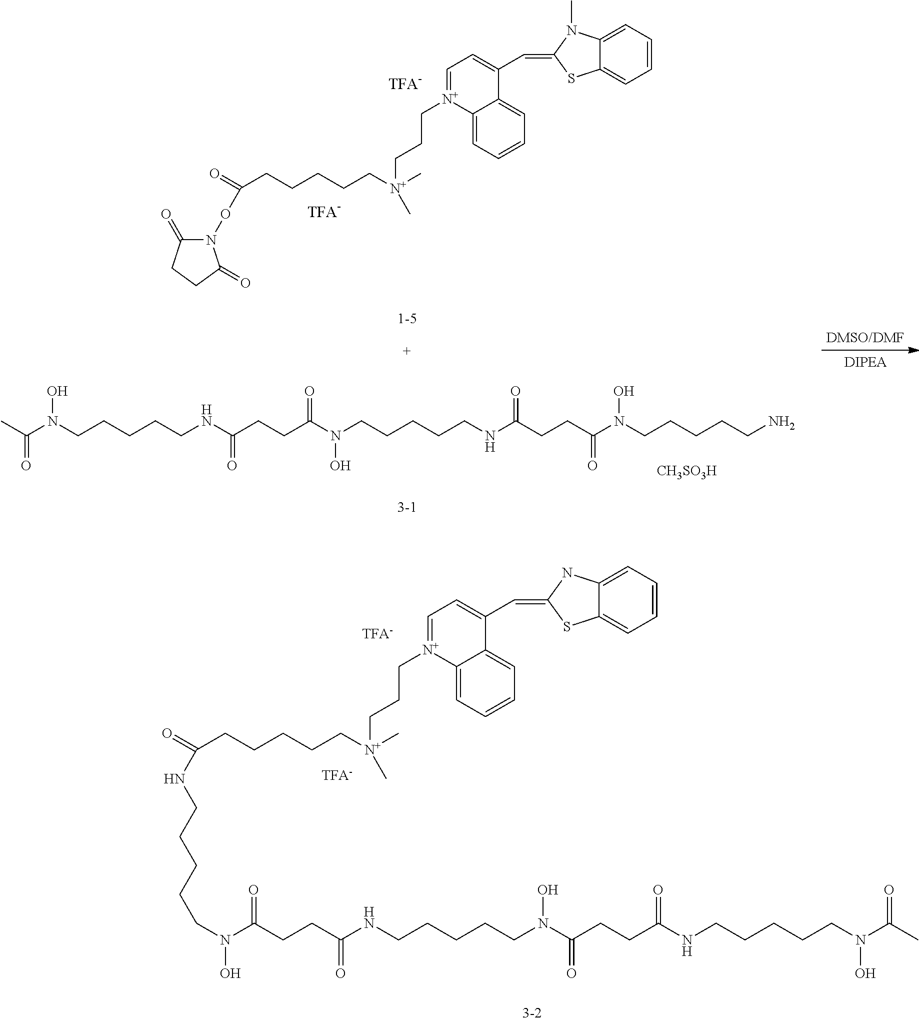

To the solution of deferoxamine mesylate (9.85 mg, 0.015 mmol) in anhydrous DMF (2 ml) and DIPEA (5.2 ul, 0.03 mmol), is added the solution of TO-NHS ester (8.14 mg, 0.01 mmol). The mixture is stirred at room temperature until TO is reacted completely. (More deferoxamine may be needed). The final product is purified by RP-HPLC C18 column.

### Example 4

### Synthesis of ¹⁸F-Nicotinic-TO

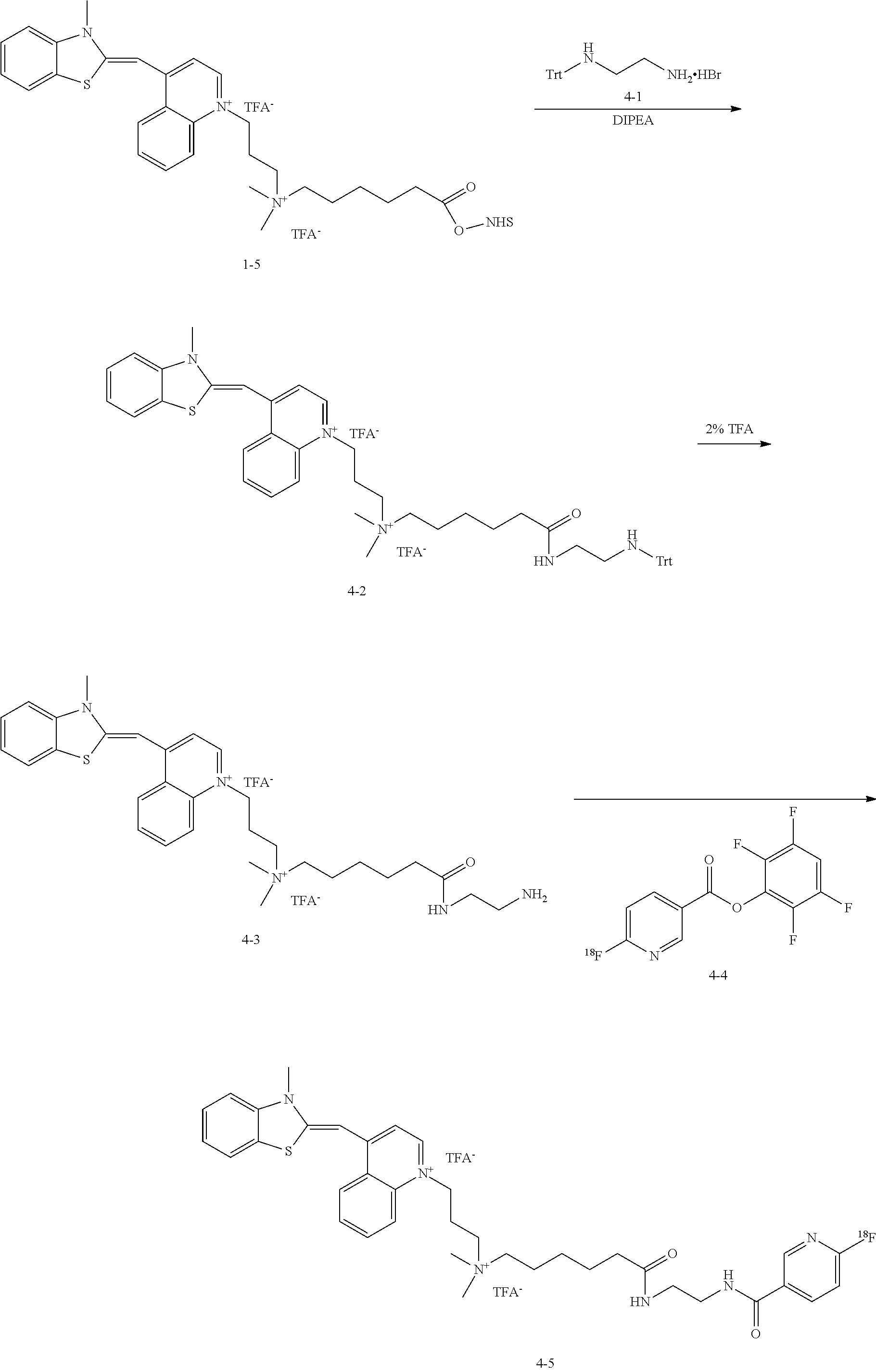

### Synthesis of Trityl-amino-TO 4-2

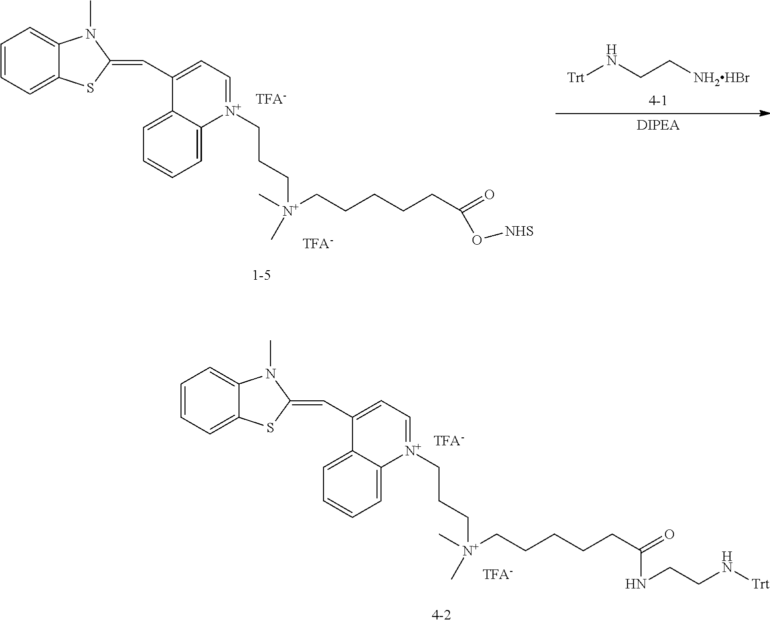

TO-Acid NHS ester 1-5 (25.8 mg, 31.7 μmol) is dissolved in DMF (600 μl). A solution of N-trityl-ethylene diamine hydrogen bromide (24.3 mg, 63.4 μmol, 2 eq) in anhydrous DMF (400 μl) and DiPEA (22 μl, 4 eq, 126 mmol) is added to the solution of NHS eater. The solution is stirred under room temperature for overnight. RP-C18 HPLC is applied for the purification (Buffer A: 0.1% TFA in water, Buffer B: 0.1% TFA and 10% water in acetonitrile) at 500 nm. Product 4-2 is collected, confirmed by MS and dried by lyophilization.

### Synthesis of Amino-TO 4-3

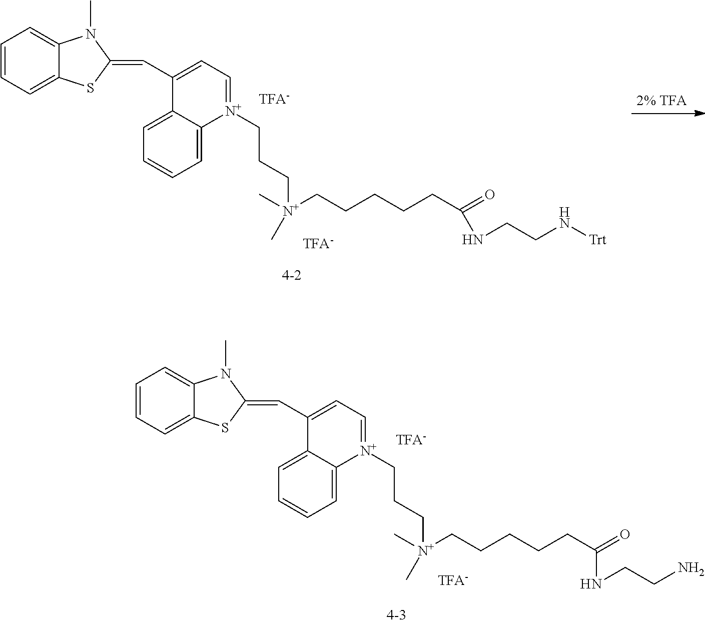

Trityl-amino-TO 4-2 (1 μmol) is dissolved in 2% TFA in acetonitrile. The reaction is monitored by HPLC or TLC. After it is complete HPLC separation is applied immediately.

### Synthesis of ¹⁸F-Nicotinic-TO 4-5

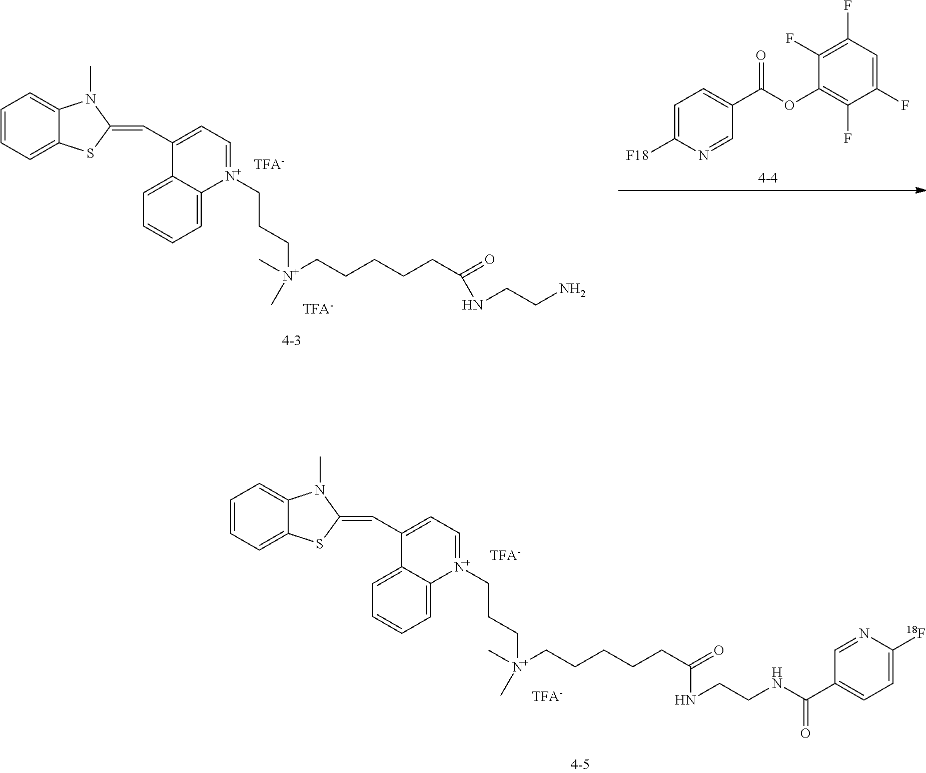

Synthesis of ¹⁸F-Nicotinic TFP ester 4-4 was taken from a known procedure (Olberg, D. E. et al. “One step radiosynthesis of 6-[(18)F]fluoronicotinic acid 2,3,5,6-tetrafluorophenyl ester ([(18)F]F-Py-TFP): a new prosthetic group for efficient labeling of biomolecules with fluorine-18” J Med Chem 53: 1732-40, 2010). Aqueous [¹⁸F]fluoride (1 mL, 100-370 MBq) was passed through an anion-exchange resin (Chromafix 30-PSHCO3, Machanery-Nagel). The [18F]fluoride was eluted off the resin to the TRACERlab reactor vessel using a mixture of 30 μL of 0.8 M aqueous solution of tetrabutylammonium bicarbonate (TBA-HCO3) in water (300 uL) and acetonitrile (300 μL). The solution was concentrated to dryness by heating at 100° C. under reduced pressure and a flow of nitrogen for 2 minutes. Acetonitrile (0.8 mL) was added twice and evaporated off as described above. To the dried TBA-[18F] complex was added N,N,N-trimethyl-5-((2,3,5,6-tetrafluorophenoxy)carbonyl)pyridin-2-aminium trifluoromethanesulfonate (9.0 mg, 19 mmol) dissolved in 1 mL of acetonitrile/tert-butanol (2:8). The sealed reaction vessel was heated to 40° C. for 10 minutes and then analyzed by radio-TLC and HPLC.

The crude reaction mixture containing [¹⁸F] 6-fluoronicotinic acid 2,3,5,6-tetrafluorophenyl ester 4-4 is diluted with 2.5 mL of water and loaded onto a preconditioned Oasis MCX Plus Sep-Pak (Waters). The cartridge is rinsed with 5 mL of water, and purified ¹⁸F-Nicotinic TFP ester 4-4 is eluted back to the reaction vessel using 2.1 mL of water/acetonitrile (3.5:6.5). Amino-TO (1.2 mg, 1.6 mmol) dissolved in 0.2M phosphate buffer (pH 7)/DMSO (1:1), 1 mL, is added to the acetonitrile/water solution of purified ¹⁸F-Nicotinic TFP ester 4-4, giving a total volume of 3.1 mL. The reaction mixture is heated to 40° C. for 15 minutes, diluted with water, and purified with preparative HPLC. The fraction containing [¹⁸F]-Nicotinic-TO 4-5 is collected, measured in a dose calibrator, and analyzed with radio-HPLC.

### Example 5

### Synthesis of ¹⁸F-PEG-TO

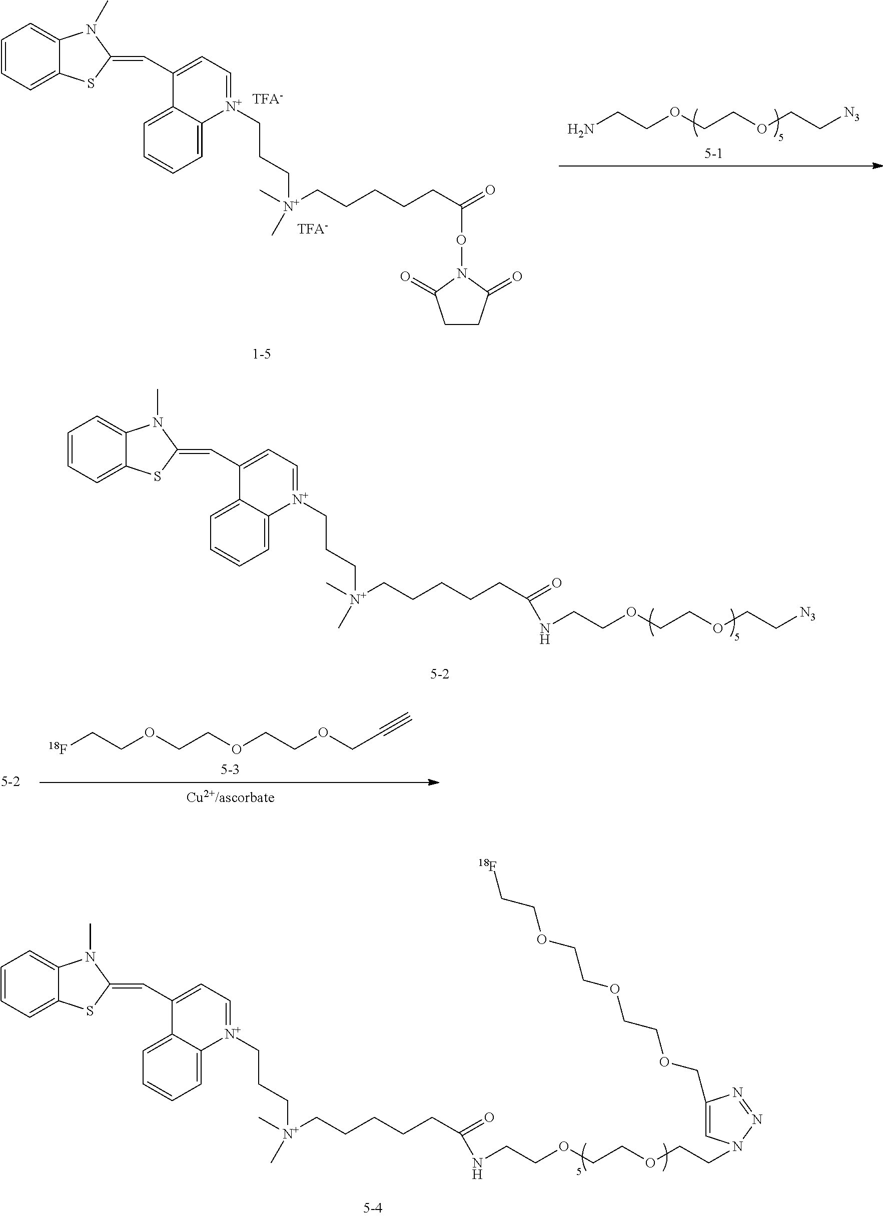

### Synthesis of Azido-TO 5-2

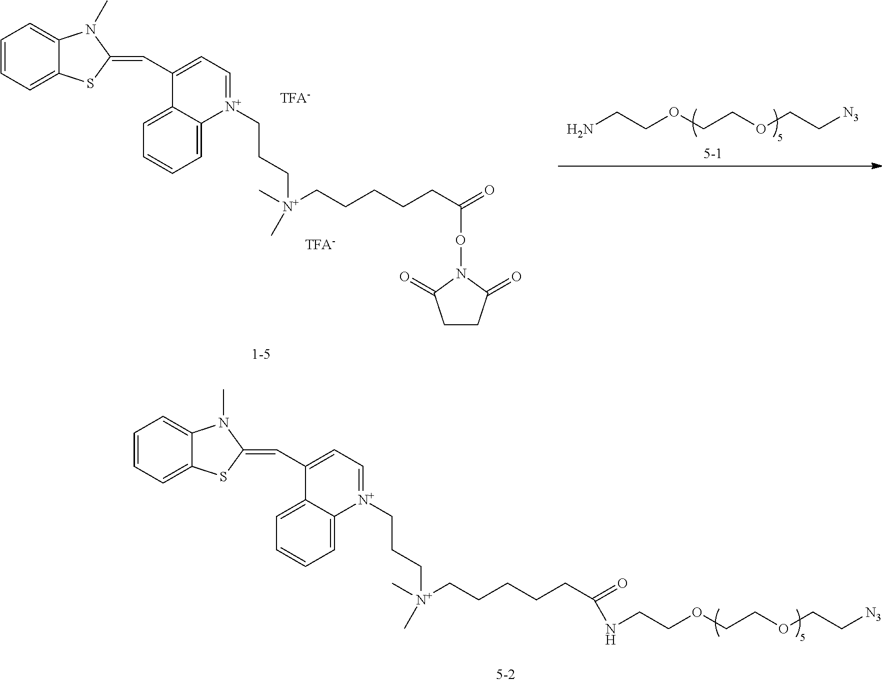

TO-acid NHS ester 1-5 (25.8 mg, 25.8/814.8=31.7 mmol) is dissolved in DMF (600 μl). A solution of azido-PEG-amine (Polypure, cat#: 12113-059, 22.2 mg, 63.4 μmol, 2 eq) in anhydrous DMF (400 μl) and DiPEA (16.6 μl, 3 eq, 95 mmol) is added to the solution of NHS eater. The solution will be stirred at room temperature overnight. RP-C18 HPLC is used for the purification (Buffer A: 0.1% TFA in water, Buffer B: 0.1% TFA and 10% water in acetonitrile) at 500 nm. The product 5-2 is confirmed by MS and dried by lyophilization.

### Synthesis of ¹⁸F-PEG-TO 5-4

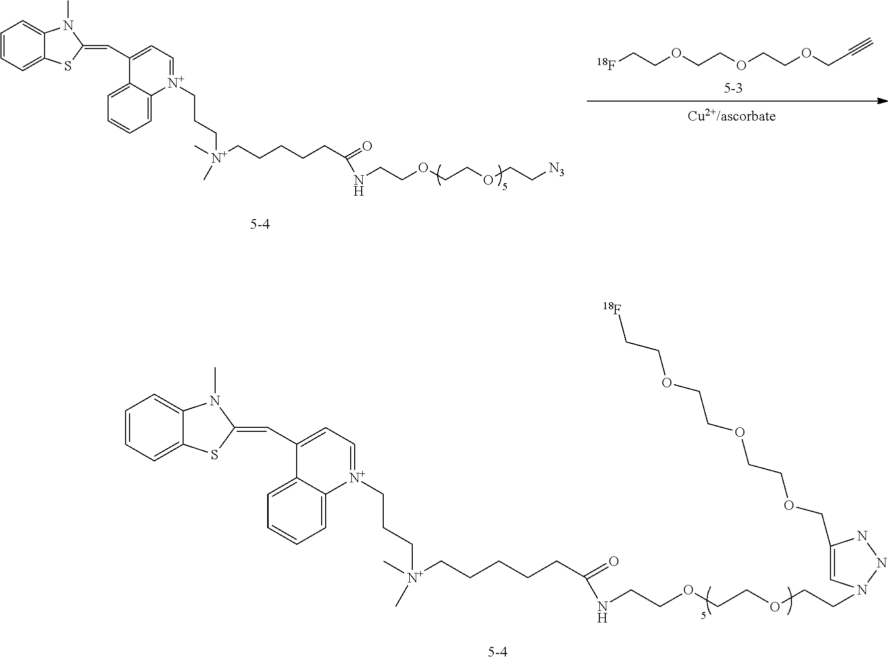

Compound 5-3 was made according to a known procedure of (Li, Z. B. et al. “Click chemistry for (18)F-labeling of RGD peptides and microPET imaging of tumor integrin alphavbeta3 expression” Bioconjug Chem 18: 1987-94, 2007). [¹⁸F] Fluoride was prepared by the 18O(p,n)¹⁸F nuclear reaction, and was then adsorbed onto an anion exchange resin cartridge. Kryptofix 222/K₂CO₃ solution (1 mL 9:1 ACN/water, 15 mg Kryptofix 222, 3 mg K₂CO₃) was used to elute the cartridge, and the resulting mixture was dried in a glass reactor. A solution of alkyne tosylate (4 mg in 1 mL ACN/DMSO) was then added, and the resulting mixture was heated at 110° C. for 15 minutes. After cooling, the reaction was quenched, and the mixture was injected onto a semipreparative HPLC for purification. The collected radioactive peak was diluted in water (10 mL) and passed through a C18 cartridge. The trapped activity was then eluted off the cartridge with 1 mL THF and used for the next reaction.

To the reactor vial with azido-TO (1 mg), 37 MBq activity, CuSO₄ (100 μL, 0.1N), and sodium L-ascorbate (100 μL, 0.3N) is added sequentially. The resulting mixture is heated at 40° C. for 20 min, and the reaction is then quenched and purified by semipreparative HPLC. The final product 18F-PEG-TO is concentrated and formulated in saline (0.9%, 500 μL) for in vivo studies.

### Example 6

### Synthesis of FH-TO

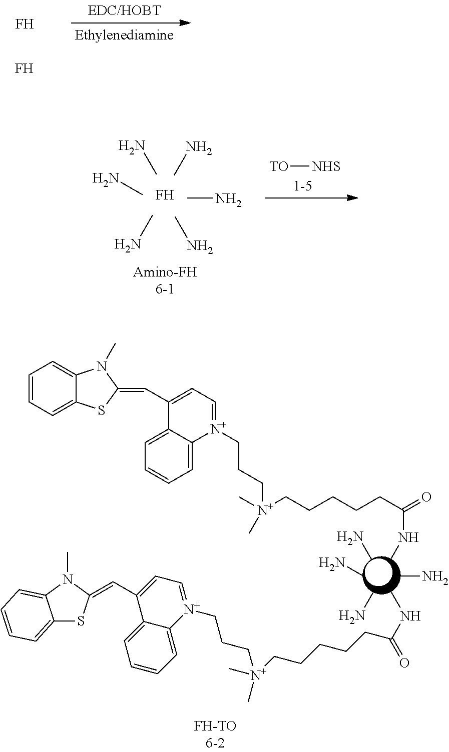

Synthesis of amino-FH, 6-1

Feraheme (30 mg Fe/mL) was exchanged into 0.1M MES buffer using gel filtration. To 1 mL (5 mg Fe, 6 umoles total COOH per mg Fe in 0.1M MES) is added as solid 25 mg of N-ethyl-N-(3-dimethylaminopropyl)carbodimide hydrochloride (MW=191, 131 umoles) and 5 mg HOBT (hydrate, MW=135, 37 μmole). The reaction mixture is incubated at room temperature for 20 minutes. To this mixture is added 35 μl of a 1M solution of ethylene diamine-HCl (Sigma) in MES (0.1M MES, pH=6.01) and the mixture heated at 50° C. for 90 minutes. The mixture is purified using a PD-10 column or concentrated and purified using a smaller NAP-5 gel filtration column. FH-amine is stored at 4° C. or stored frozen.

### Synthesis of FH-TO 6-2

To a solution of amino-FH (6-1, 1 ml, 5 mg Fe) is added TO-NHS 1-5 (1 mg, 0.125 μmol). The solution is incubated at room temperature for 2 hours. FH-TO is purified by gel filtration using Sephadex G-25.

### Example 7

### Synthesis of CLIO-TO

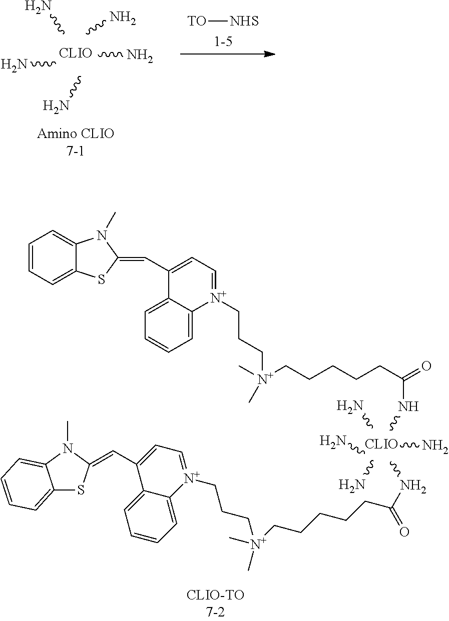

Amino-CLIO is prepared by known methods (Palmacci, S. et al. (1993) in U.S. Pat. No. 5,262,176; Josephson, L. et al. “High-efficiency intracellular magnetic labeling with novel superparamagnetic-Tat peptide conjugates” Bioconjug Chem 10: 186-91, 1999). The preparation of CLIO-TO 7-2 is carried out as for FH-TO 6-2 in Example 6.

### Example 8

### Synthesis of Gado-DTPA-YO

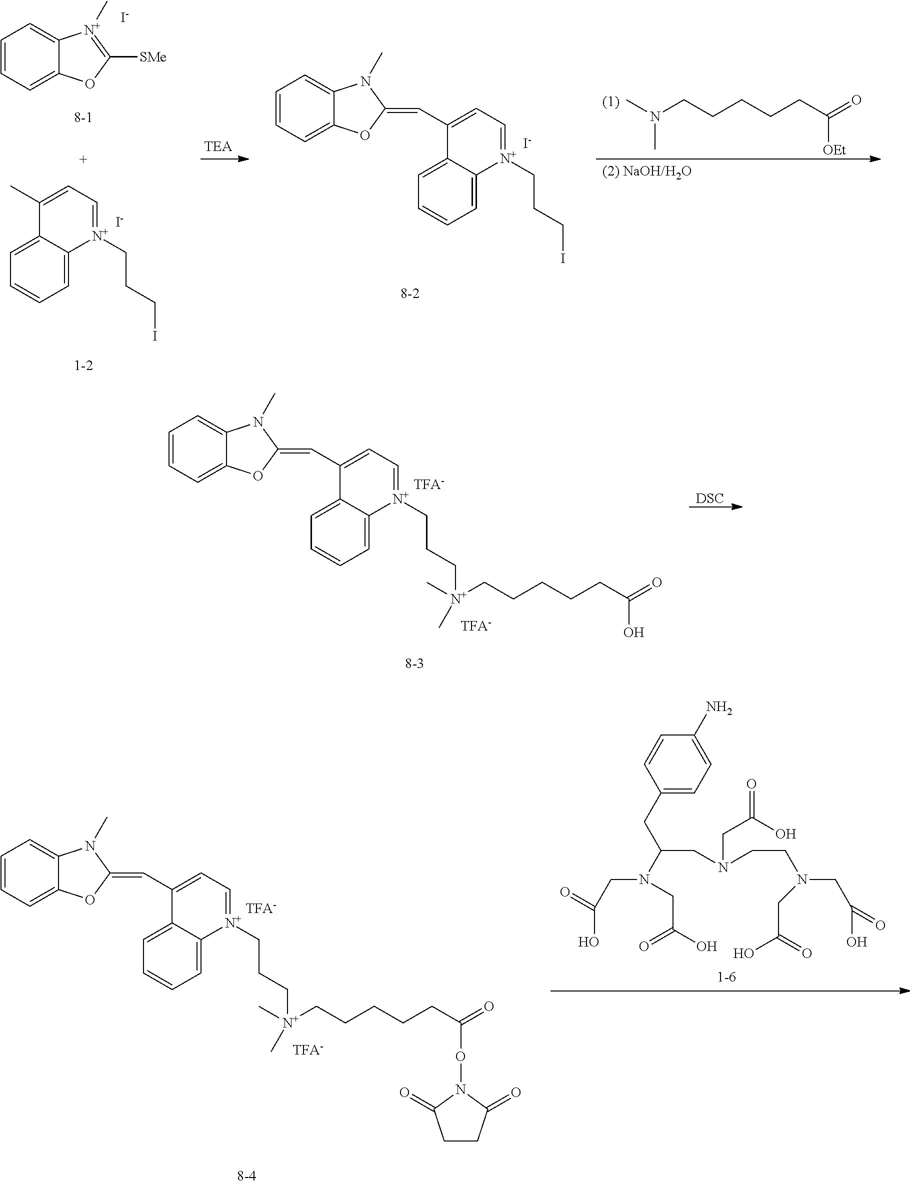

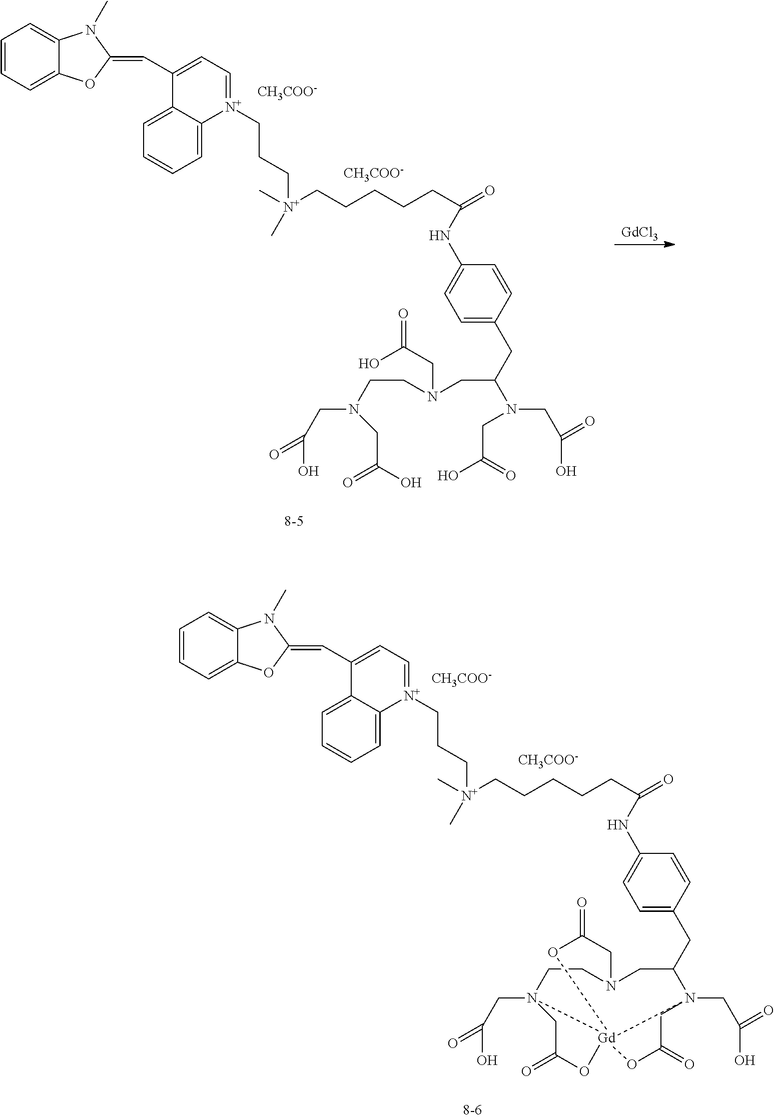

### Synthesis of YO-Iodide 1-3

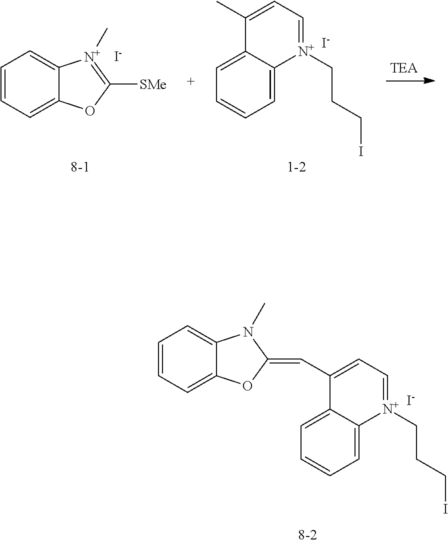

Compound 8-1 was synthesized according to a reported procedure (Thompson, M. “Synthesis, photophysical effects, and DNA targeting properties of oxazole yellow-peptide bioconjugates” Bioconjug Chem 17: 507-13, 2006). The synthesis was performed in a two-step reaction by first mixing 2-mercaptobenzoxazole (3, 2.0 g, 11 mmol), iodomethane (8.0 mL, 55 mmol), and K₂CO₃ (1.0 g, 7 mmol) in 100 mL of acetone for 60 minutes at 70° C. Reaction mixture was filtered hot, and the filtrate was dried in vacuo. Recrystallization was performed using ethanol:diethyl ether which provided 1.3 g (40%) of pure 2-methylmercaptobenzoxazole iodide. Next, 2-methylmercapto-benzoxazole (1.0 g, 3 mmol) and trimethyloxonium tetrafluoroborate (1.1 g, 3.3 mmol) were mixed in 50 mL of dichloromethane in a nitrogen environment for 5 hours at room temperature. Precipitation with diethyl ether was followed by filtration and yielded 3.5 g (90%) of pure 2-methylmercapto-3-methylbenzoxazole. MALDITOF gave a molecular weight (m/z) of 179.8 (180.2, calculated for C9H10NOS+). 1H NMR (400 MHz, DMSO-d6) ä 3.31 (s, 3H, SCH3), 3.91 (s, 3H, NCH3), 7.50-7.61 (m, 1H, Ar), 7.84-7.88 (m, 1H, Ar), 8.00 (d, J) 8.82 Hz, 1H, Ar), 8.61 (d, J=7.66 Hz, 1H, Ar).

An alternate synthesis of 2-methylmercapto-3-methylbenzoxazole was performed by refluxing 2-methylmercaptobenzoxazole (4, 1.0 g, 3 mmol) in p-methyltoluenesulfonate (10 mL, 66 mmol) under nitrogen for 60 minutes. The reaction mixture was allowed to cool to room temperature, filtered, and washed with diethyl ether. Recrystallization from ethanol:diethyl ether gave 1.5 g (70%) of pure 2-methylmercapto-3-methylbenzoxazole tosylate. MALDI-TOF gave a molecular weight (m/z) of 180.5 (180.2, calculated for C9H10NOS+).

### Synthesis of Oxazole Yellow 8-2

1-(3-iodopropyl)-4-[(3-methylbenzoxazol-2(3H)-ylidene)-methyl]quinolinium 8-2 is synthesized by mixing 4-methyl-1-(3-iodopropyl)quinolinium iodide (1-2, 3 mmol) and 2-methylmercapto-3-methylbenzoxazole (8-1, 3 mmol) in 30 mL of absolute ethanol with slight heating for 5 minutes. To this solution, triethylamine (840 μl, 6 mmol) is added causing the reaction mixture to immediately turn a deep red color. Heating is discontinued and the reaction mixture is allowed to stir for 60 minutes. Product 8-2 is precipitated by the addition of 150 mL of diethyl ether.

### Synthesis of YO-Acid 8-3

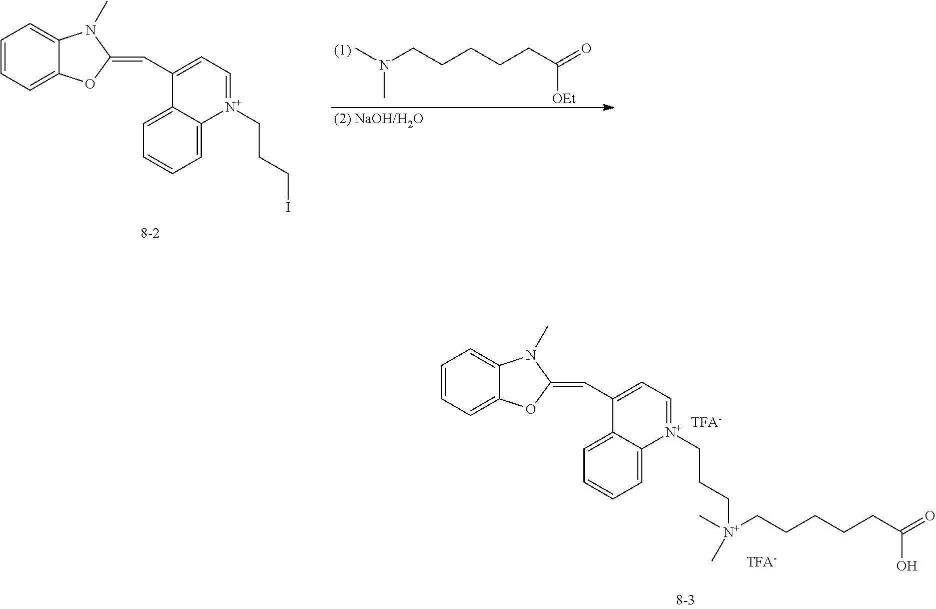

In a sealed tube, compound 8-2 (341.1 mmol) is suspended and dissolved into 10 mL anhydrous ethanol. A solution of ethyl N,N-dimethyl-6-aminohexanoate (320 mg, 1.709 mmol, 5 equiv.) in 2 mL anhydrous ethanol is added. The reaction mixture is stirred overnight at 145° C. The solution is cooled to room temperature and the precipitate formed is separated by filtration, washed with ether and dried under vacuum. The solid powder is dissolved in 8 mL of a mixture of acetonitrile/water (50:50). A solution of NaOH 1M (4 mL) is added. The reaction mixture is stirred at room temperature for 1 hour and purified by RP-HPLC (UV monitoring at 480 nm). The fraction collected is lyophilized.

### Synthesis of YO Acid NHS Ester 8-4

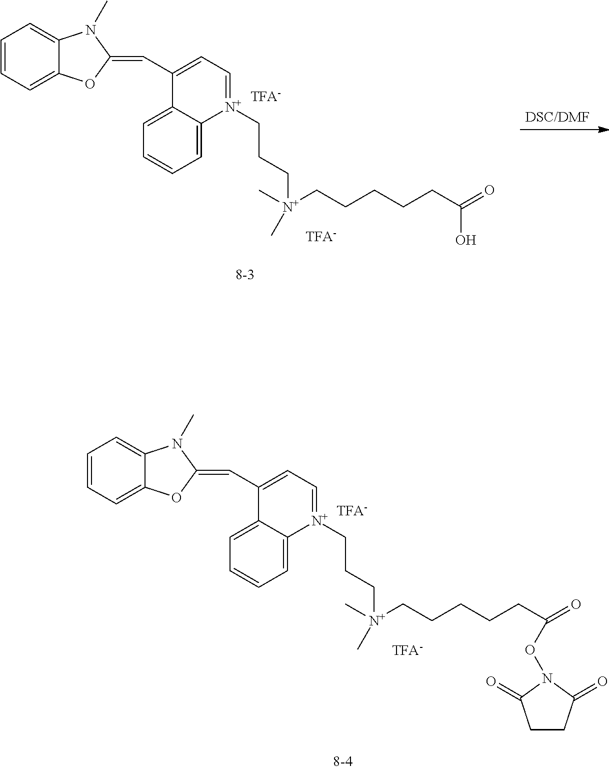

Compound 8-3 (156.5 μmol) is dissolved in 1.3 mL anhydrous DMF Anhydrous DIPEA (70 μL, 401.9 μmol, 2.6 equiv.) and di(N-succinimidyl) carbonate (101.2 mg, 395.1 μmol, 2.5 equiv.) are added. The reaction mixture is stirred at room temperature for 1 hour. Precipitation from ether affords the succinimidyl ester intermediate as a solid.

### Synthesis of YO-DTPA 8-5

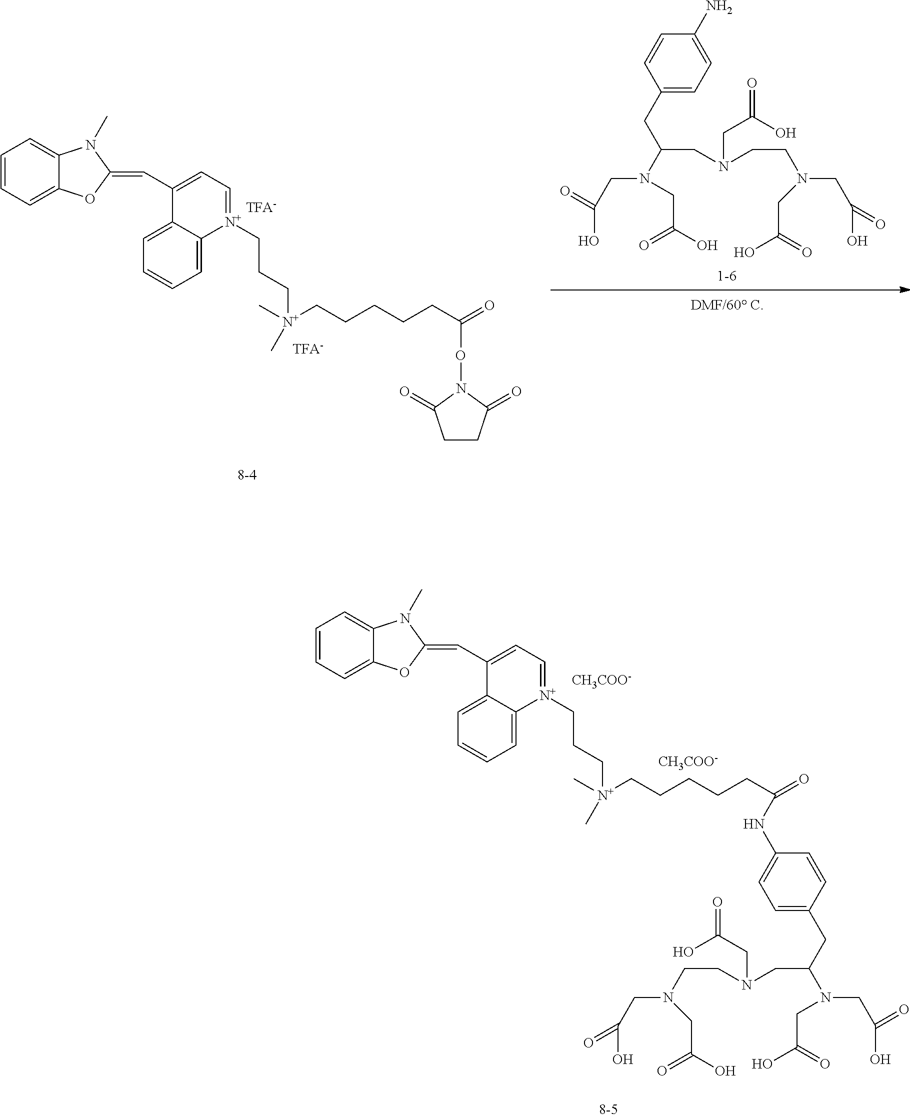

In a polypropylene vial, p-NH2-Bn-DTPA (70.4 mg, 141.2 μmol) is dissolved in 706 μL anhydrous DMF containing DIPEA (295 μL, 1.694 mmol, 12.0 equiv.). A solution of the succinimidyl ester intermediate 8-4 (78.3 μmol, 0.55 equiv.) in 330 μL anhydrous DMF is added. The reaction mixture is stirred overnight at 60° C. and purified by RP-HPLC (UV monitoring at 480 nm). The fraction collected is lyophilized to obtain compound 8-5.

### Synthesis of Gado-DTPA-YO 8-6

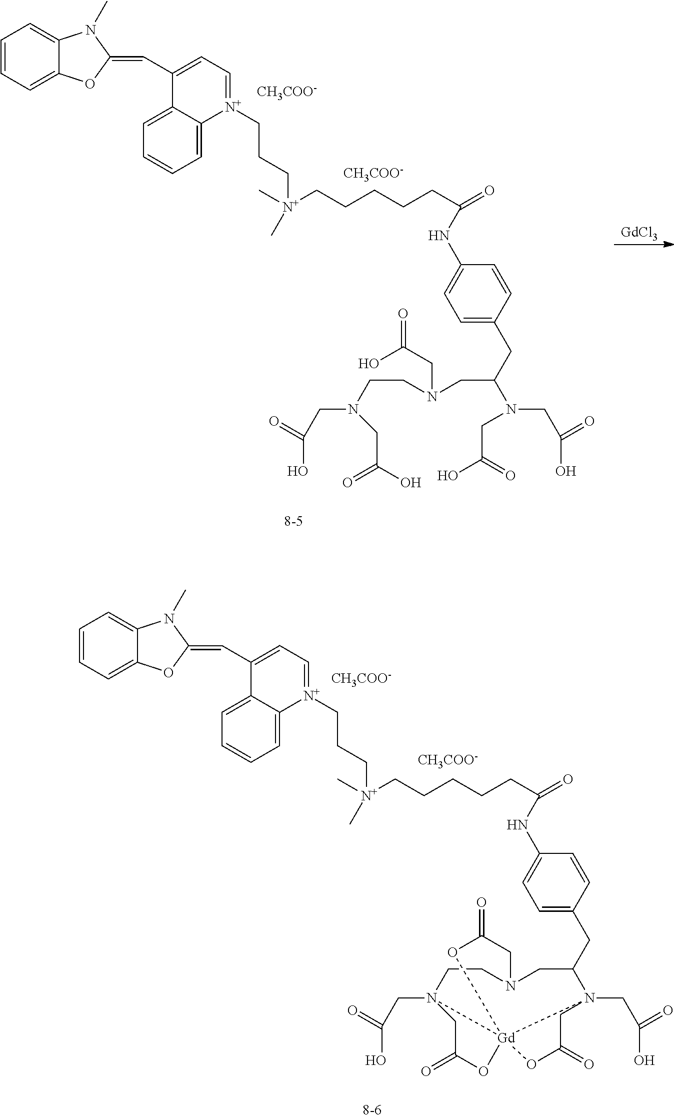

Compound 8-5 (24.5 μmol) is dissolved in 900 μL of a solution of citric acid 0.1 M pH 5.7. GdCl₃.6H₂O (37.0 mg, 99.5 μmol, 4.1 equiv.) dissolved in 1.8 mL citric acid 0.1 M pH 5.7 is added. The reaction mixture is stirred for 2 hours at room temperature and purified by RP-HPLC (UV monitoring at 480 nm). The fraction collected is lyophilized.

### Example 9

### Synthesis of Gado-DOTA-YO 9-2

### Synthesis of DOTA-YO 9-1

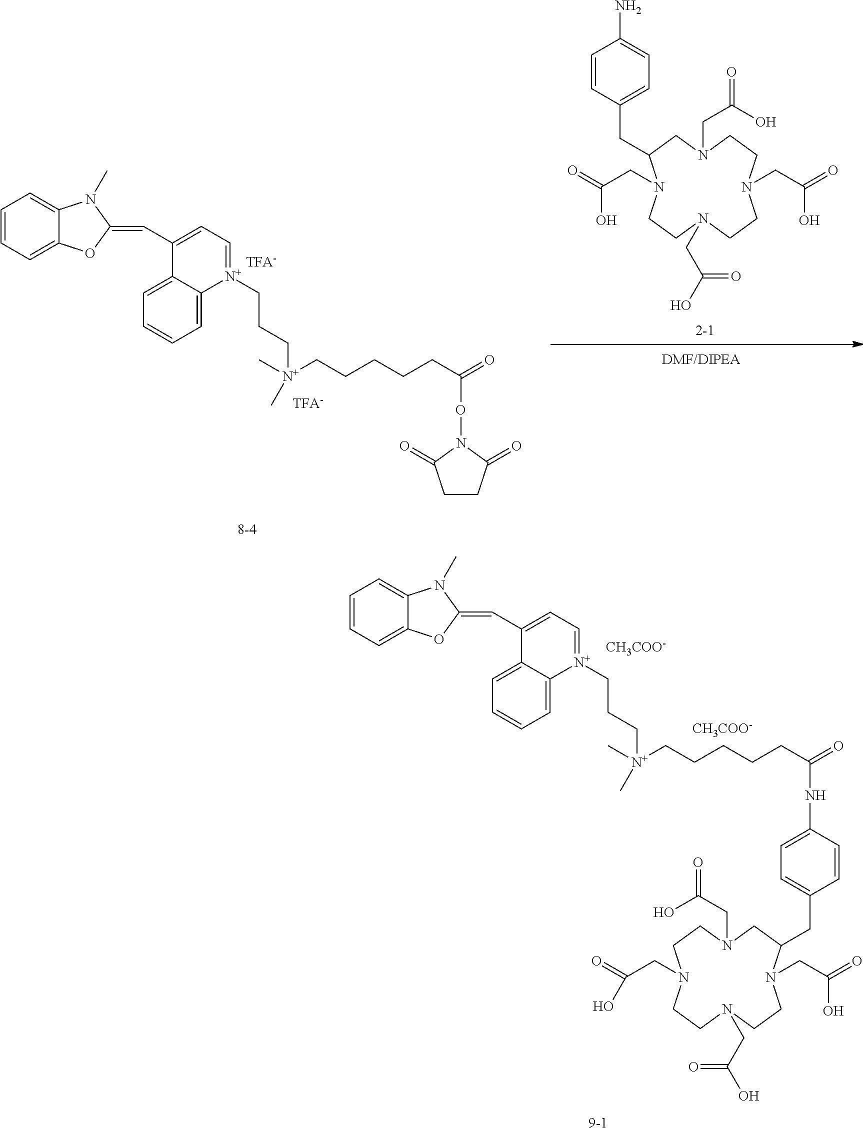

In a polypropylene vial, p-NH2-Bn-DOTA (141.2 μmol) is dissolved in 706 L anhydrous DMF containing DIPEA (295 μL, 1.694 mmol, 12.0 equiv.). A solution the succinimidyl ester intermediate 8-4 (78.3 μmol, 0.55 equiv.) in 330 μL anhydrous DMF is added. The reaction mixture is stirred overnight at 60° C. and purified by RP-HPLC (UV monitoring at 480 nm). The fraction collected is lyophilized. Compound 9-1 is obtained.

### Synthesis of Gado-DOTA-YO 9-2

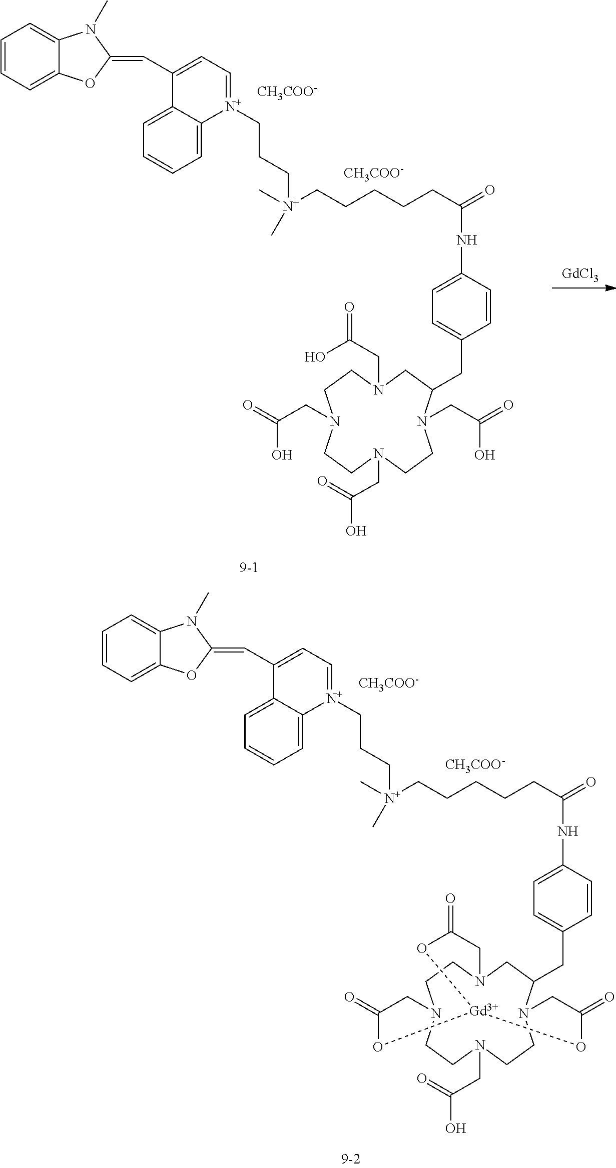

Compound 9-1 (24.5 μmol) is dissolved in 900 μL of a solution of citric acid 0.1 M pH 5.7. GdCl₃.6H₂O (37.0 mg, 99.5 μmol, 4.1 equiv.) dissolved in 1.8 mL citric acid 0.1 M pH 5.7 is added. The reaction mixture is stirred for 2 hours at room temperature and purified by RP-HPLC (UV monitoring at 480 nm). The fraction is lyophilized.

### Example 10

### Synthesis of Gado-PI 10-7

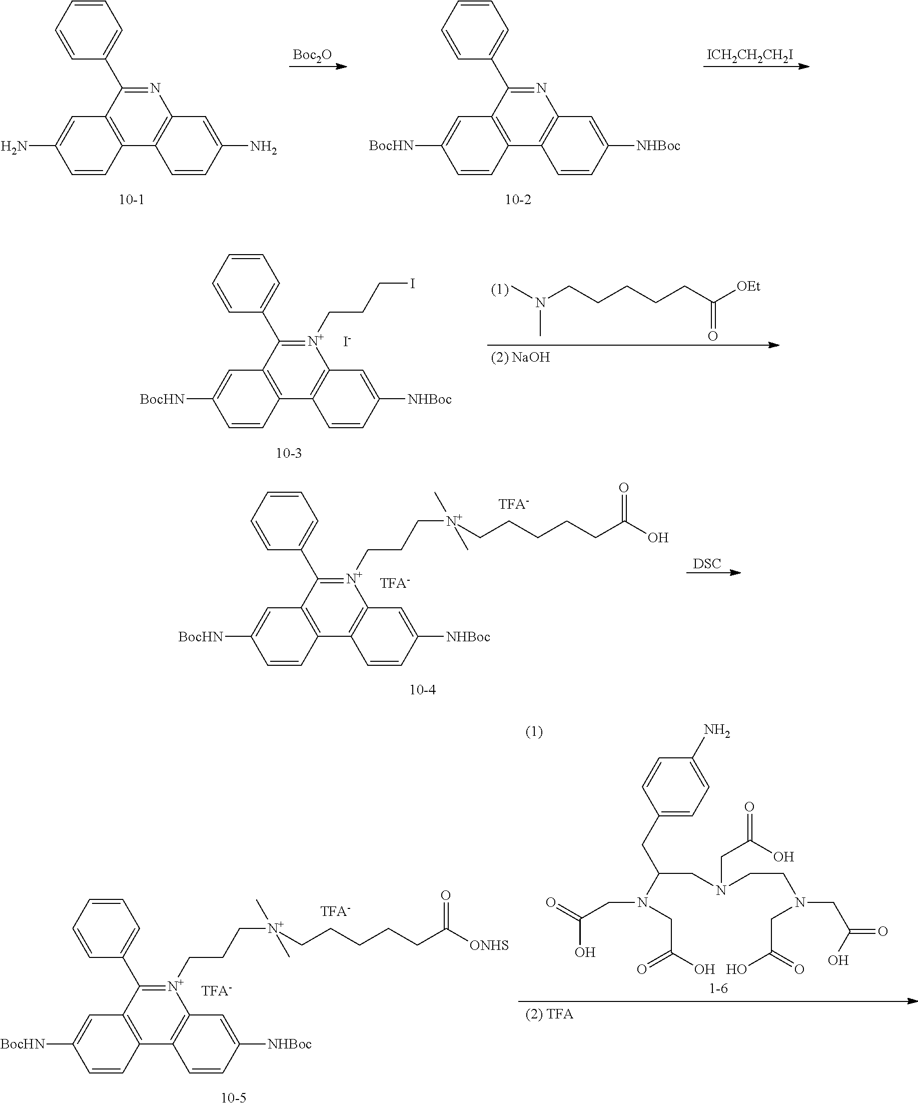

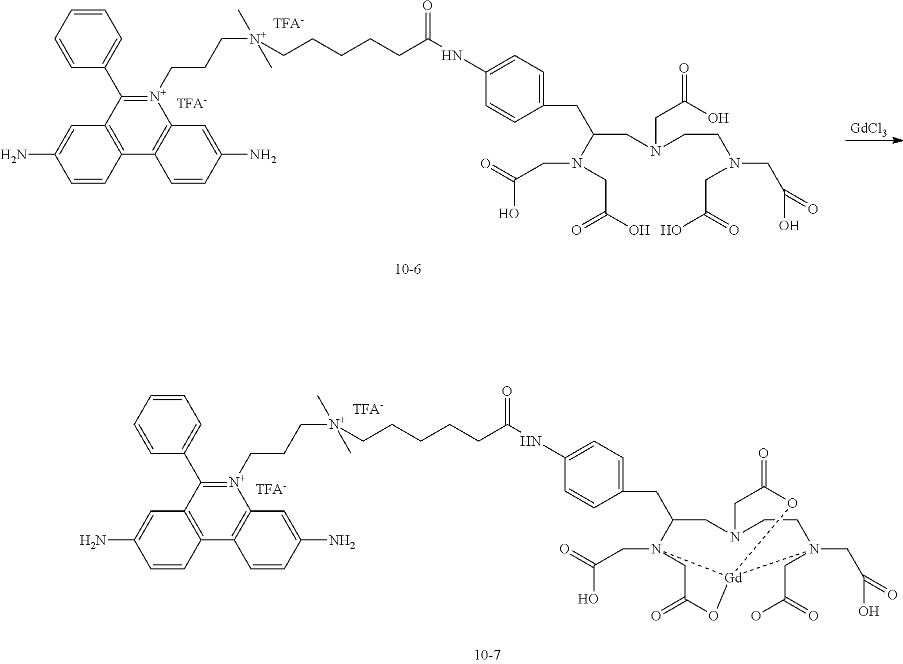

### Synthesis of Boc Protected PI, 10-2

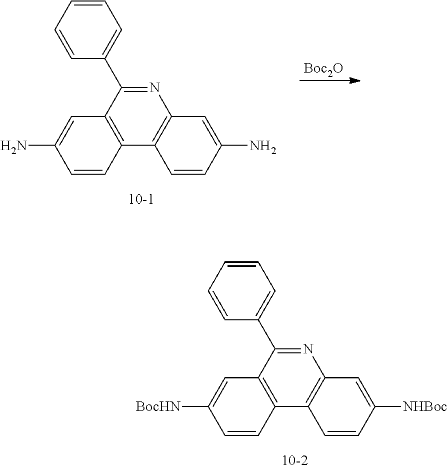

2.7-Diamino-9-phenylphenanthridine (285 mg, 1 mmol) in DMF or pyridine (2 ml) is treated with Boc anhydride (2.2 eq.) in the presence of DIPEA (2.2 eq.)(no need for base if pyridine used as the solvent) under the flow of Ar. The mixture is stirred at room temperature overnight. The mixture is poured into water (10 ml) and the solid is collected by filtration. The product 10-2 is purified by HPLC. This procedure is modified from a known procedure (Watkins, T. I. “Trypanocides of the phenanthridine series: 1. The effect of changing the quaternary grouping in dimidium bromide” J Chem. Soc., 3059-3064, 1952).

### Synthesis of Iodo-PI 10-3

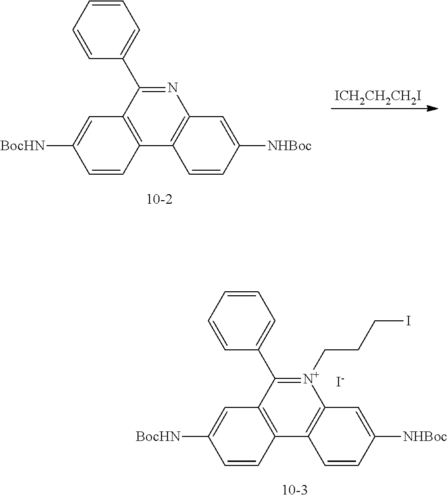

Compound 10-2 (1.36 mmol) is refluxed with 50 mL of 1,3-diiodopropane for 6 hours. After cooling, the reaction mixture is filtered and iodo-PI is crystallized from methanol or other solvent.

### Synthesis of PI-Acid 10-4

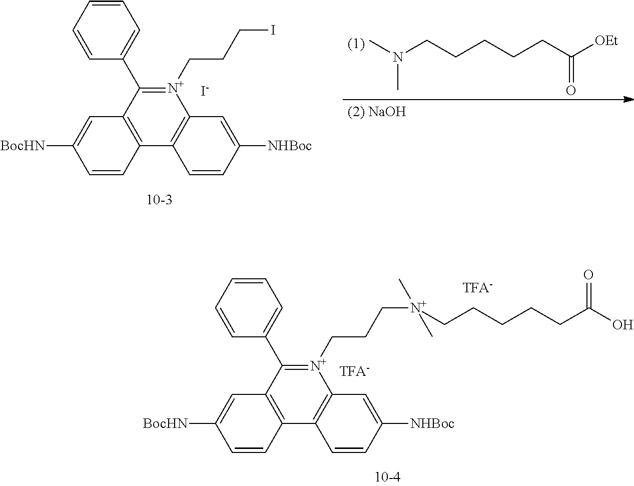

In a sealed tube, compound 10-3 (0.341 mmol) is suspended and dissolved into 10 mL anhydrous ethanol. A solution of ethyl N,N-dimethyl-6-aminohexanoate (320 mg, 1.709 mmol, 5 equiv.) in 2 mL anhydrous ethanol is added. The reaction mixture is stirred overnight at 145° C. The solution is cooled down to room temperature. The precipitate formed is separated by filtration, washed with ether and dried under vacuum. The solid powder is dissolved in 8 mL of a mixture of acetonitrile/water (50:50). A solution of NaOH 1M (4 mL) was added. The reaction mixture is stirred at room temperature for 1 h and purified by RP-HPLC. Compound 10-4 is obtained as solid powder after lypholization.

### Synthesis of PI-NHS Ester 10-5

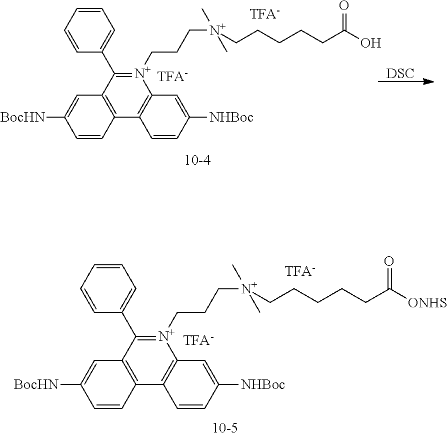

Compound 10-4 (0.15 mmol) is dissolved in 1.3 mL anhydrous DMF Anhydrous DIPEA (2.6 equiv.) and di(N-succinimidyl) carbonate (2.5 equiv.) are added. The reaction mixture is stirred at room temperature for 1 hour. Precipitation from ether affords the succinimidyl ester intermediate 10-5 as a solid.

### Synthesis of DTPA-PI 10-6

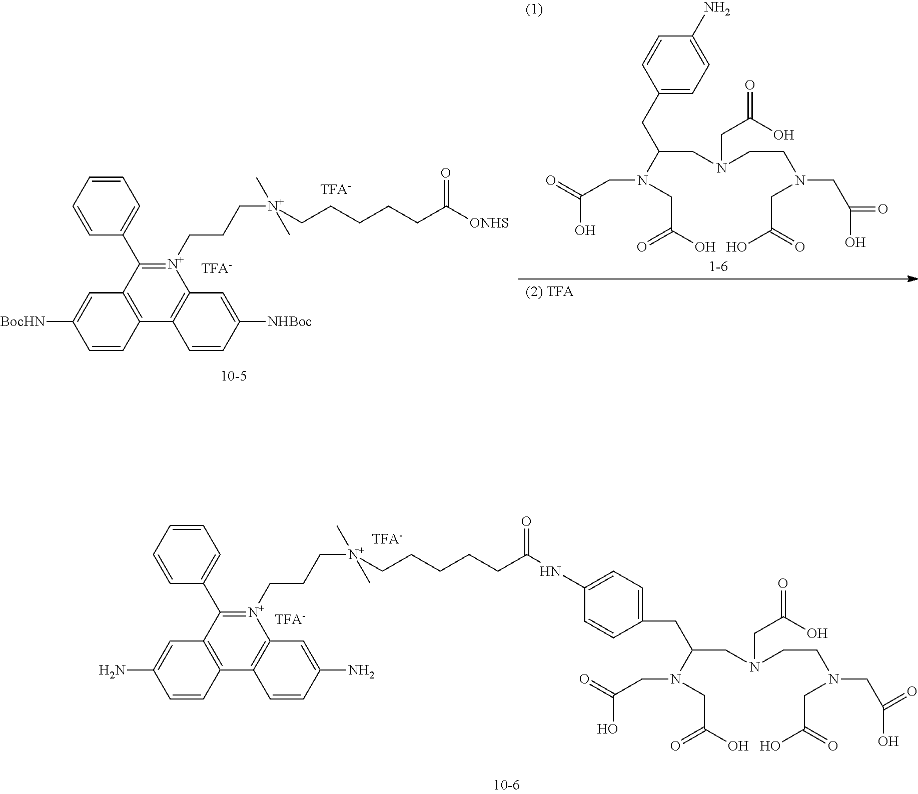

In a polypropylene vial, p-NH₂-Bn-DTPA (70.4 mg, 141.2 μmol) is dissolved in 706 μL anhydrous DMF containing DIPEA (295 μL, 1.694 mmol, 12.0 equiv.). A solution the succinimidyl ester intermediate 10-5 (0.55 equiv.) in 330 μL anhydrous DMF is added. The reaction mixture is stirred overnight at 60° C. and purified by RP-HPLC. The fraction is lyophilized. The Boc-protected precursor is re-dissolved in dichloromethane and TFA 1:1 (v/v). The mixture is stirred at room temperature until all the reaction is complete. The solvent is evaporated and the residue is purified by RP-HPLC.

### Synthesis of Gado-PI 10-7

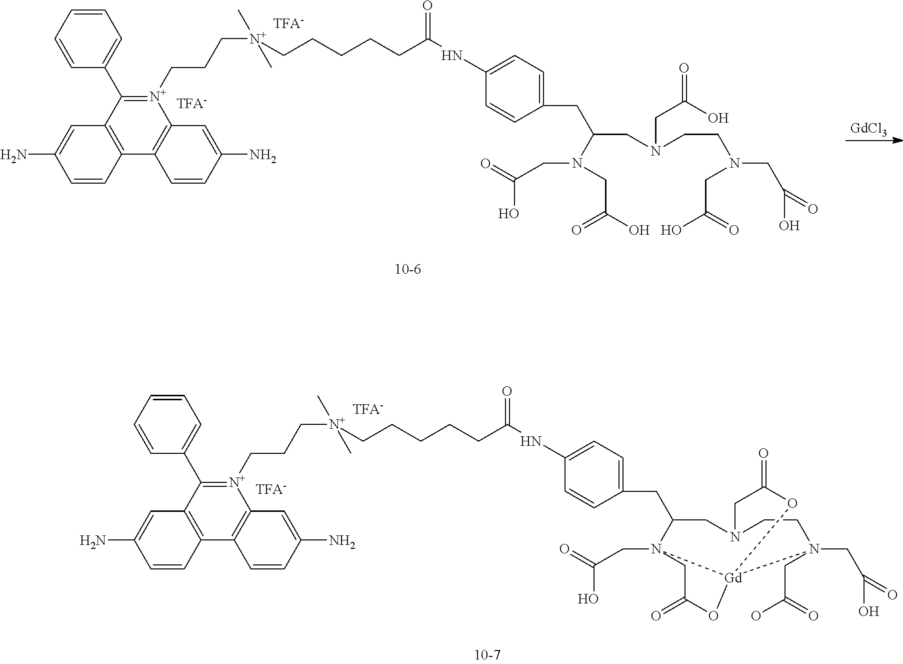

Compound 10-6 (25 μmol) is dissolved in 900 μL of a solution of citric acid (0.1 M pH 5.7). GdCl₃.6H₂O (37.0 mg, 99.5 μmol, 4.1 equiv.) dissolved in 1.8 mL citric acid (0.1 M pH 5.7) is added. The reaction mixture is stirred for 2 hours at room temperature and purified by RP-HPLC. The fraction collected is lyophilized. Compound 10-7 (GadoPI) is obtained as solid powder.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition for imaging dead or dying cells, the composition comprising a vital fluorochrome conjugate comprising a vital fluorochrome, a reporter group, and a linker that connects the fluorochrome to the reporter group.

2. The composition of claim 1, wherein the vital fluorochrome comprises an aliphatic arm attached to an unsaturated ring system at a first end of the aliphatic arm, and a quaternary, positively charged nitrogen attached to the aliphatic arm at a second end.

3. The composition of claim 1, wherein the vital fluorochrome conjugate comprises a compound of Formula I: 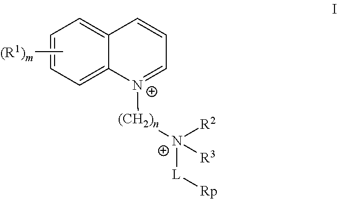
wherein:
L is a linker and comprises a C₂₋₂₀ alkyl chain, wherein any of the carbons in the C₂₋₂₀ alkyl chain can be replaced with —C(O)—, —C(O)₂, O, S, S(O), S(O)₂, —NR^{a}—, —NR^{a}(O)—, or a triazole;
Rp is a reporter group;
R¹ is aryl or heteroaryl optionally substituted by aryl or heteroaryl; or 1 or 2 R¹ adjacent to each other and together with the carbon atoms to which they are attached form 1 or 2 aryl or heteroaryl rings, optionally substituted by 1, 2, 3, or 4 substituents independently selected from OR^{a} and —NR^{a}₂;
R² and R³ are independently selected from H and C₁₋₆ alkyl; or
R² and R³ are each independently L-Rp;
R^{a} is selected from H and C₁₋₆ alkyl; and
m and n are each independently 0, 1, 2, 3, 4, 5, 6, 7, 8, 9, or 10.

4. The composition of claim 1, wherein the reporter group is a metal chelator that chelates a detectable metal atom.

5. The composition of claim 4, wherein the metal is paramagnetic or radioactive.

6. The composition of claim 4, wherein the metal is selected from gadolinium, dysprosium, ¹¹¹indium, ⁹⁹mTc, ⁶⁴copper, and ⁶⁸gallium.

7. The composition of claim 4, wherein the reporter group is

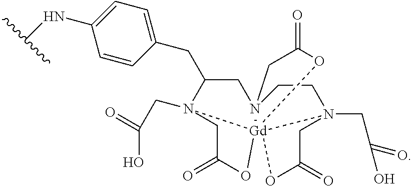

8. The composition of claim 1, wherein the reporter group comprises a radioactive halide.

9. The composition of claim 8, wherein the radioactive halide is ¹⁸Fluorine.

10. The composition of claim 8, wherein the reporter group is

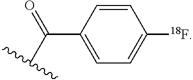

11. The composition of claim 1, wherein the compound is selected from:

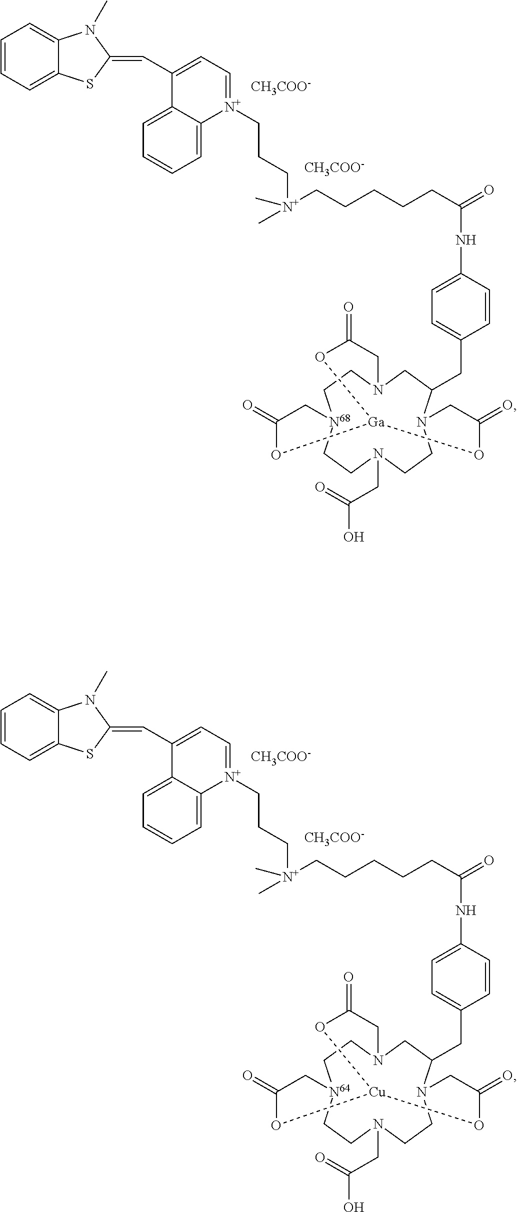

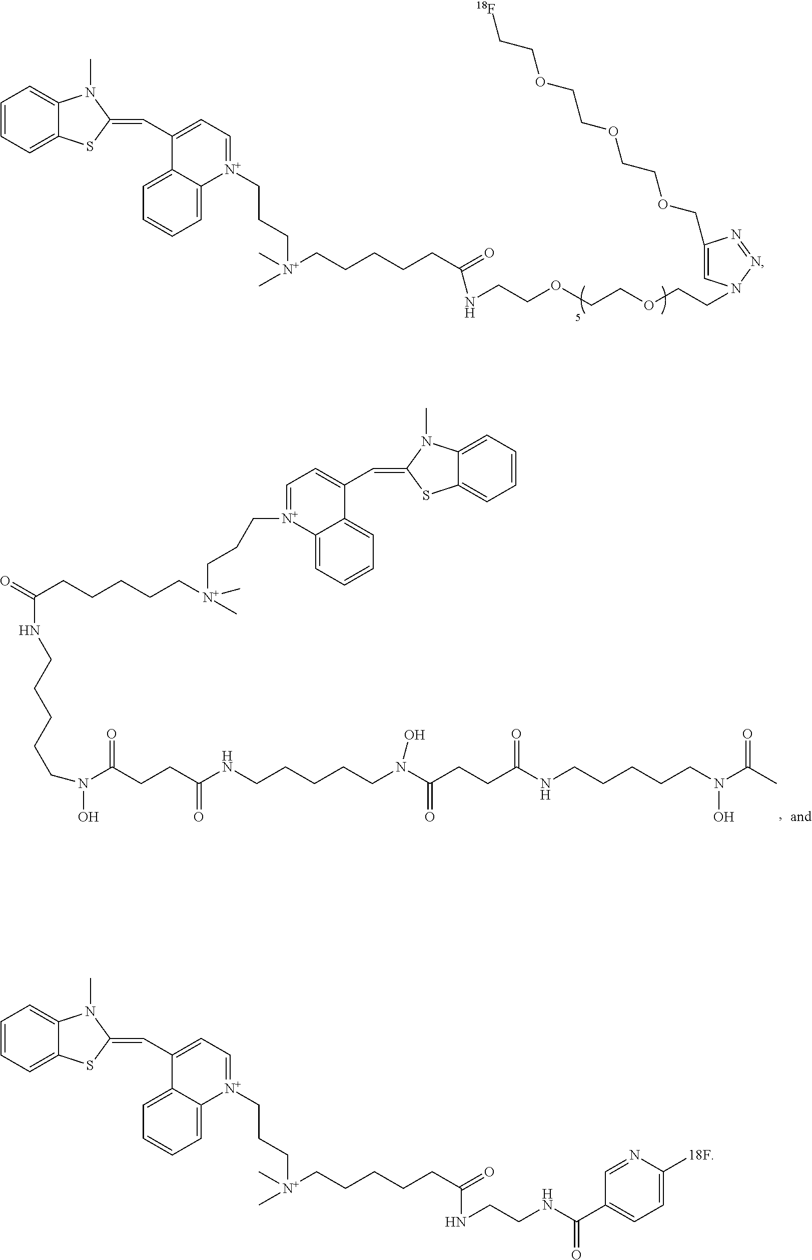

12. A method of imaging dead or dying cells in a subject, the method comprising:
administering to the subject a vital fluorochrome conjugate comprising a vital fluorochrome, a reporter group, and a linker that connects the vital fluorochrome to the reporter group, for a time sufficient for the vital fluorochrome conjugate to enter dead or dying cells and bind to nucleic acids; and
obtaining an image of the vital fluorochrome conjugate in the subject.

13. The method of claim 12, wherein the image is obtained by a single photon emission computed tomography (SPECT) scan, a positron emission tomography (PET) scan, or a magnetic resonance imaging (MRI) scan.

14. The method of claim 12, wherein the subject is a mammal.

15. The method of claim 12, wherein the dead or dying cells are cancer cells treated by chemotherapy.

16. The method of claim 12, wherein the dead or dying cells are cells from an organ transplanted into the subject.

17. A method of detecting dead or dying cells in myocardium tissue in a subject, the method comprising:
administering to the subject a vital fluorochrome conjugate comprising a vital fluorochrome, a reporter group, and a linker that connects the vital fluorochrome to the reporter group, for a time sufficient for the vital fluorochrome conjugate to enter the dead or dying myocardial cells and bind to nucleic acids;
and obtaining an image of the vital fluorochrome conjugate in the subject.

18. The method of claim 17, wherein the tissue is ischemic due to a myocardial infarction.

19. The method of claim 18, wherein the dead or dying cells are imaged between 1 day and 10 days after the myocardial infarction occurs.

20. A process for preparing a compound of Formula I: 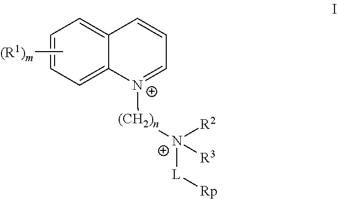
wherein:
L is a linker and comprises a C₂₋₂₀ alkyl chain, wherein any of the carbons in the C₂₋₂₀ alkyl chain can be replaced with —C(O)—, —C(O)₂, O, S, S(O), S(O)₂, —NR^{a}—, —NR^{a}C(O)—, or a triazole;
Rp is a reporter group capable of attaching to a detectable atom;
R¹ is aryl or heteroaryl optionally substituted by aryl or heteroaryl; or 2 or 3 R¹ adjacent to each other and together with the carbon atoms to which they are attached form 1 or 2 aryl or heteroaryl rings, optionally substituted by 1, 2, 3, or 4 substituents independently selected from —OR^{a} and —NR^{a}₂;
R² and R³ are independently selected from H and C₁₋₆ alkyl;
R^{a} is selected from H and C₁₋₆ alkyl; and
m and n are each independently 0, 1, 2, 3, 4, 5, 6, 7, 8, 9, or 10;
the process comprising:
reacting a compound of Formula I-(1): 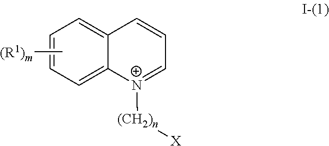
wherein:
X is a halide;
with an amine of Formula I-(2): 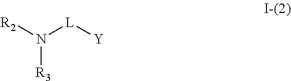
wherein:
Y is selected from 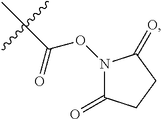
—NH₂, N₃, and 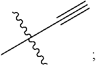
to provide a compound of Formula I-(3): 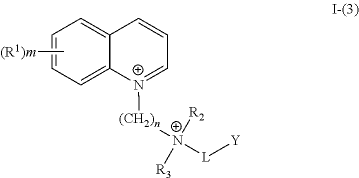
and reacting the compound of Formula I-(3) with a compound of Formula I-(4): 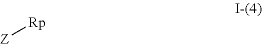
wherein:
Z is selected from 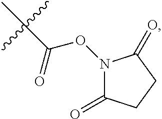
—NH₂, N₃, and 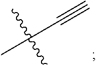
to produce the compound of Formula I.

21. The process of claim 20, wherein Y is N₃ and Z is

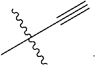

22. The process of claim 20, wherein Y is 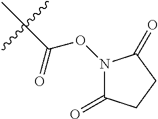
and Z is NH₂.
